(19) United States
(12) Patent Application Publication
(10) Pub. No.: US 2024/0025970 A1
(43) Pub. Date: Jan. 25, 2024

(54) LUNG TREATMENT COMPOSITIONS

(71) Applicants: Sherwood Russ Lehrman, Los Altos, CA (US); Ehud Goldin, Sunnyvale, CA (US)

(72) Inventors: Sherwood Russ Lehrman, Los Altos, CA (US); Ehud Goldin, Sunnyvale, CA (US)

(21) Appl. No.: 18/249,237

(22) PCT Filed: Oct. 17, 2021
(86) PCT No.: PCT/US2021/055333

§ 371 Date: Apr. 14, 2023

Related U.S. Application Data
(60) Provisional application No. 63/092,625, filed on Oct. 16, 2020.

(51) Int. Cl. C07K 14/785; A61K 31/685; A61K 38/45; A61K 45/06; A61K 47/64; A61K 9/14; A61P 31/12; A61P 11/00

(52) U.S. Cl. CPC C07K 14/785; A61K 31/685; A61K 38/45; A61K 45/06; A61K 47/64; A61K 9/14; A61P 31/12; A61P 11/00; C12Y 207/10002

(57) ABSTRACT

Described are heat stable protein-phospholipid concentrates within lung surfactant therapeutic compositions for the treatment of respiratory diseases in premature infants, children and adults. The concentrates can be suitable for localized delivery with improved transport to and spreadability within the lung and other tissues resulting in greater efficacy and efficiency of treatment.

### CROSS-REFERENCE TO RELATED APPLICATION

This application claims priority to U.S. Provisional Patent Application No. 63/092,625, filed Oct. 16, 2020 which is incorporated herein by reference in its entirety.

### FIELD

The present teachings relate to a composition for lung surfactants, a method using the composition in treating respiratory diseases, pulmonary injury and infection, a method of making a therapeutic lung surfactant composition (LSC) and a kit for both economic treatment and efficient transport of the LSC therapeutic.

### BACKGROUND

Respiratory diseases affect both premature infants and adults. In premature infants, as commonly seen in the NICU, the infant is diagnosed with Infant Respiratory Distress Syndrome (IRDS) also known as neonatal respiratory distress syndrome (NRDS) and was initially known as hyaline membrane disorder. It is a consequence of lung development immaturity as the lungs are structurally unable to produce sufficient levels of pulmonary surfactant that support independent breathing until around 35 weeks gestational age (GA). Injury resulting from the treatment of the premature infant with supplemental oxygen, ventilator-induced lung injury (VILI), can result in NRDS, lead to inflammation resulting in bronchopulmonary dysplasia (BPD) and bacterial infection leading to late onset sepsis. In the 1990s the use of animal-derived lung surfactant products and less aggressive oxygen supplementation shifted the primary cause of BPD from ventilation-induced lung injury to inflammation of immature lung tissue from exposure to oxygen.

In adults, adult respiratory distress syndrome (ARDS) most often occurs due to inflammation and subsequent degradation of lung tissue. These tissues become permeable which facilitates increased plasma in the lung. The plasma inhibits the biological actions of lung surfactant. Additional illnesses, such as COPD and emphysema are likely due to chronic inflammation. The adult respiratory distress syndromes can be a result of acute lung injury (ALI), ventilator-induced lung injury (VILI), and systemic inflammatory response syndrome (SIRS) from physical insults to the lung tissues including shock, bacterial, viral and nosocomial pneumonias, and inhalation of toxic gases, vapors, fumes and particles and injuries such as mechanical ventilation, administration of oxygen, aspiration, and intubation.

Lung surfactant is a mixture of protein and phospholipid that facilitates lung inhalation and exhalation at the gas/liquid interface of the interior of alveoli sacs within the deepest recesses of the lung where circulating blood cells carry oxygen from the lungs and return carbon dioxide for exhalation. Both hydrophilic and hydrophobic regions are found within the proteins and lipids of the surfactant. Dipalmitoylphosphatidylcholine (DPPC), the main phospholipid component of lung surfactant, reduces surface tension by positioning hydrophilic head groups in the aqueous-based alveolar fluid and hydrophobic tails facing towards the air where gas exchanges occur by adsorbing oxygen at the air-water interface of alveoli.

There are four surfactant proteins identified in air-breathing mammals, Surfactant protein A (SP-A, SFTPA1, SFTP1) and Surfactant protein D (SP-D, SFTPD, SFTP4) are water-soluble and have collagen-like domains. Both SP-A and SP-D are part of the innate immune system's collectins.

Surfactant protein B (SP-B, SFTP3, SFTB3) is a lung surfactant needed for lung expansion and compression. It is lipid-associated and assists the transport of lipid molecules into and out of alveolar membrane tissue. Surfactant protein C is an integral membrane protein that is also found in lung surfactant and is regulated by SP-B.

Current therapeutics to replace deficient mammalian lung surfactant in pre-term infants suffering from the respiratory diseases described above are derived from minced bovine and porcine lung and calf lung lavage. A synthetic lung surfactant, CHF5633 contains SP-B and SP-C analogs and may provide an alternative lung surfactant therapeutic. However, animal derived lung surfactant has several concerns ranging from variable composition, risk of contamination by animal pathogens, poor heat stability, and mishandling resulting in failure to adequately resuspend the therapeutic during administration.

Thus, there remains an unmet need for a lung surfactant composition resistant to elevated temperature exposures, remains in a suspended state, free of the potential for animal pathogen contaminants and of a consistent, immune tolerant composition.

Therefore, Applicant has developed in various embodiments of the disclosed innovations and claimed inventions, a heat stable lung surfactant protein-lipid composition concentrate for the treatment of respiratory disease suitable for localized delivery and that can be used to help the spread and transport of therapeutics to the lung that address these unmet needs.

### INCORPORATION BY REFERENCE

All publications, references, patents, and patent applications mentioned in this document are herein incorporated by reference to the same extent as if each individual publication, reference, patent, or patent application was specifically and individually indicated to be incorporated by reference. In the event of inconsistent usages between this document and those documents so incorporated by reference, the usage in the incorporated reference should be considered supplementary to that of this document; for irreconcilable inconsistencies, the usage in this document controls.

Reference will now be made to various embodiments, examples of which are illustrated in the accompanying drawings.

### BRIEF DESCRIPTION OF THE DRAWINGS

FIG. 1 depicts the sequence of parent SP-B.

FIG. 2 depicts the sequence of synthetic Monomeric-SP-B.

FIG. 3 depicts the sequence of structure of Monomeric-SP-B.

FIG. 4 depicts the protected, synthesized Monomeric SP-B peptide prior to cleavage from the resin and removal of protecting groups.

FIG. 5 illustrates the cleavage reaction of the fragment 2425 peptide.

FIG. 6 is the HPLC profile of SP-B Fragment 2425 material is shown in FIG. 5.

FIG. 7 illustrates the synthesis scheme for Fragment 212223.

FIG. 8 is the HPLC-UV-MS analysis of purified fragment 212223 is illustrated in FIG. 7.

FIG. 9 illustrates the synthesis scheme for synthesis of peptide 212223-2425.

FIG. 9A depicts the assemble of the five fragments and FIG. 9B illustrates the chemical reaction to join the fragments and the resulting disulfide bonds.

FIG. 10 is the HPLC profile of Cys-Protected SP-B Fragment 2122232425-3.

FIG. 11 is the 3HPLC Profile of Monomeric SP-B Fragment 2122232425.

FIG. 12 graphically illustrates the dynamic surface tension of Monomeric SP-B peptide in the presence of phospholipid.

FIG. 12A illustrates the minimum surface tension and FIG. 12B illustrates the maximum surface tension as droplest were alternatively constrained and expanded.

FIG. 13 presents SP-B and SP-C amino acid sequences of lung surfactant proteins as disclosed in various embodiments described and claimed herein.

### DETAILED DESCRIPTION OF VARIOUS EMBODIMENTS

Before the present compositions and methods are described, it is to be understood that the disclosed invention is not limited to particular compositions, methods, kits and experimental conditions described, as such compositions, methods, kits and conditions may vary. It is also to be understood that the terminology used herein is for purposes of describing particular embodiments only, and is not intended to be limiting.

What is disclosed herein are various embodiments to reiterate the unique aspects the disclosed innovation provides for improved storage, shipment, and economical treatment of a mammal suffering from deficient production of lung surfactant and/or dysfunctional production lung surfactant.

### Definitions

Unless defined otherwise, all technical and scientific terms used herein have the same meaning as commonly understood by one of ordinary skill in the art to which this invention belongs. As used herein, the recited terms have the following meanings. The following definitions are included to provide a clear and consistent understanding of the specification and claims. Any methods and materials similar or equivalent to those described herein can be used in the practice or testing of the disclosed invention, as it will be understood that modifications and variations are encompassed within the spirit and scope of the instant disclosure.

References in the specification to “one embodiment, “an embodiment,” another embodiment,” and the like, indicate that the described embodiment can include a particular aspect, feature, structure, moiety, or characteristic, but every embodiment may not necessarily include the particular aspect, feature, structure, moiety, or characteristic. Moreover, such phrases are not necessarily referring to the same embodiment. Further, when a particular aspect, feature, structure, moiety, or characteristic is described in connection with an embodiment, it is submitted that it is within the knowledge of one of ordinary skill in the art to effect or connect such aspect, feature, structure, moiety, or characteristic in connection with other embodiments whether or not explicitly described.

It is further noted that the claims may be drafted to exclude an optional element. As such, this statement is intended to serve as antecedent basis for the use of exclusive terminology, such as “solely,” “only,” “other than”, and the like, in connection with any element described herein, and/or the recitation of claim elements or use of “negative” limitations.

The term “and/or” means any one of the items, any combination of the items, or all of the items with which this term is associated. The phrases “one or more” and “at least one” when read in context of its usage are readily understood by one of skill in the art, particularly. For example, the phrase can mean one, two, three, four, five, six, ten, 100, or any upper limit approximately 10, 100, or 1000 times higher than a recited lower limit.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range as if each numerical value and sub-range is explicitly recited. As a non-limiting example, each range discussed herein can be readily broken down into a lower third, middle third and upper third, as well as nested ranges within a larger range, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges.

For example, a range of “about 1% to about 5%” or “about 0.1% to 5%” should be interpreted to include not just about 1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The term “about” as used herein can allow for a degree of variability in a value or range, for example, within 10%, within 5%, or within 1% of a stated value or of a stated limit of a range. For example, “about 50” percent can in some embodiments carry a variation from 45 to 55 percent. In yet another example, “about 10.0 wt. %” can be between 9.5 wt. % and 10.5 wt. %.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

For integer ranges, the term “about” can include one or two integers greater than and/or less than a recited integer at each end of the range. Unless indicated otherwise herein, the term “about” is intended to include values, e.g., ratios, mg/kg, weight percentages, proximate to the recited range that are equivalent in terms of the functionality of the individual ingredient, element, the composition, or the embodiment. The term about can also modify the end-points of a recited range as discuss above in this paragraph.

In this document, the terms “a,” “an,” or “the” are used to include one or more than one unless the context clearly dictates otherwise. Thus, for example, a reference to “a component” includes a plurality of such components, so that a component Z includes a plurality of components Z. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting; Information that is relevant to a section heading may occur within or outside of that particular section.

In the methods or processes described herein, the steps can be carried out in any order without departing from the principles of the invention, except when a temporal or operational sequence is explicitly recited.

Furthermore, specified steps can be carried out concurrently unless explicit claim language recites that they be carried out separately. For example, a claimed step of doing A and a claimed step of doing B can be conducted simultaneously within a single operation, and the resulting process will fall within the literal scope of the claimed process.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “amorphous powder” as used herein can refer to, the transformation of a wet material, e.g., a liquid, solution, suspension, emulsion, liposome and the like to a dry, microparticulate material by subjecting the wet material to at least one drying method including, but not limited to, spray drying, supercritical fluid freezing, bubble drying, and lyophilization as is known to the skilled artisan. The amorphous powder can be any non-crystalline solid matrix. The solid matrix can be a powder, tablet powder within a cake, tablet, and dispersed w/in a capsule.

The term “attached” as used herein can refer to, for example, one protein joined with another protein, one molecule joined with another molecule and/or protein, peptide, amino acid sequence, and migrating together, for example, across a lipid bi-layer. The joined molecules, proteins, and the like can have a linking amino acid sequence, a mutual chemical bond including but not limited to a covalent bond, hydrophobic bonding or hydrophilic interactions connecting the molecules, proteins and the like together.

The term “bioactive agent” as used herein can refer to a natural or synthetic moiety, substance, chemical, therapeutic and the like which can impart a biological function, activity or property. Example include but are not limited to: an anti-inflammatory drug, tincture, extract, anti-infective drugs, bronchodilation drugs, antihistamines, cyclooxygenase inhibitors, leukotriene antagonists, PLA2 inhibitors, PAF antagonists and prophylactics of asthma, antiallergics, bronchodilators, lung surfactants, analgesics, antibiotics, antibacterials, antifungals, antivirals, antiprotozoans, leukotriene inhibitors or antagonists, antihistamines, decongestants and anti-tussive drug substances, anticholinergics, β-blockers, adrenergic, β₂-adrenoreceptor agonist, anesthetics, anti-tuberculars, imaging agents, cardiovascular agents, enzymes, steroids, genetic material, nucleic acid vectors, antisense agents, proteins, peptides and combinations thereof.

The term “complex” as used herein can refer e.g. to one protein associated with another protein and migrating together, for example, across a lipid bi-layer. The attached molecules, proteins, and the like lack a mutual chemical bond including but not limited to a covalent bond, hydrophobic bonding or hydrophilic interactions joining the molecules, proteins and the like together.

The term “deficient” as used herein can refer to an inadequate production of e.g., lung surfactant, a lung surfactant protein, a lipid, phospholipid. Deficient surfactant can be associated with respiratory distress syndrome (RDS), restrictive respiratory diseases as well as premature neonates with immature lung development.

The term “emulsion” as used herein can refer to a mixture, multiphasic biphasic solution, of two or more liquids that are normally immiscible, one dispersed in the other.

The term “fused” and “fusion” are used interchangeably and as used herein can refer to the attachment of a bioactive agent including, but not limited to, an anti-inflammatory agent, anti-infective agent to either the N- or C-terminus of SP-C by, in a non-limiting example, a covalent bond.

The phrases “percent homology,” “% homology,” “percent identity,” or “% identity” refer to the percentage of sequence similarity found in a comparison of two or more amino acid or nucleic acid sequences. Percent identity can be determined electronically, e.g., by using the MEGALIGN program (LASERGENE software package, DNASTAR). The MEGALIGN program can create alignments between two or more sequences according to different methods, e.g., the Clustal Method. (Higgins, D. G. and P. M. Sharp (1988) Gene 73:237-244.) The Clustal algorithm groups sequences into clusters by examining the distances between all pairs. The clusters are aligned pairwise and then in groups. The percentage similarity between two amino acid sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no homology between the two amino acid sequences are not included in determining percentage similarity. Percent identity between nucleic acid sequences can also be calculated by the Clustal Method, or by other methods known in the art, such as the Jotun Hein Method. (See, e.g., Hein, J. (1990) Methods Enzymol. 183:626-645.) Identity between sequences can also be determined by other methods known in the art, e.g., by varying hybridization conditions.

Nucleic acid and amino acid sequences having at least 85%, 86%, 87%, 88%, 89%, 90%, 91%, 92%, 93%, 94%, 95%, 96%, 97%, 98%, and 99% sequence identity to the disclosed nucleic acid and amino acid sequences for use in the claimed lung surfactant composition are also envisioned herein.

The term “lipid” as used herein can refer to a natural and a synthetic saturated fatty acid, an unsaturated fatty acid, a fatty acid sugar, waxes, sterols, a phospholipid, phosphoglycerolipids, sphingolipids and digalactosylglycerolipids as is known to the skilled artisan.

The term “liposome” as used herein can refer to an oil in water or water in oil emulsion. The complexing with one another of fatty acids and proteins can also be termed a liposome.

The terms “infant,” “neonatal infant,” “neonate” and “newborn” are used interchangeably and as used herein can refer to a baby in the first 28 days after birth. The terms can apply to premature, full term and postmature infants.

The term “lung surfactant” as used herein can refer to biocompatible mixtures of protein and lipid that facilitate lung compliance. The protein can be naturally occurring, recombinant, homolog, analog, mimic, modified and synthetic. The lipid can be naturally occurring, homolog, analog, mimic, modified and synthetic.

The term “lung surfactant complex” as used herein can refer to a mixture of at least one lung surfactant protein and at least one lipid which can form at least one of a suspension and an emulsion.

The term “lung surfactant composition” (LSC)” as used herein can refer to a lung surfactant complex that can optionally contain at least one of an excipient, water, a buffer, and a bioactive agent. The bioactive agent can be included in the lung surfactant complex's mixture to form at least one of a suspension and an emulsion or alternatively added to the complex's suspension or emulsion. In a various embodiment of the disclosed innovations and claimed inventions, the bioactive agent can be fused, attached or complexed with a lung surfactant protein.

The term “suspension” as used herein can refer to a solid including, but not limited to, an amorphous powder, dispersed in a liquid such as an aqueous solution, dilute organic solution and buffering medium. Suspension can also refer to the resulting mixture of one or more lung surfactant protein(s) with one or more lipid(s).

The term “glass transition temperature (T_{g})” as used herein can refer to the transition within an amorphous particle, material, lacking an ordered arrangement (opposite of a crystal solid which has an ordered structure), from a dry, hard, “glassy” material to a viscous or rubbery state when exposed to temperature increase(s). In other words, the T_{g} indicates the temperature where the amorphous material transitions to a more viscous state. Such a transition can be associated with a breakdown in an amorphous molecule's structure and thus a loss of the molecule's stability and spreadability which can result in diminished efficacy and efficiency when the amorphous molecule is a pharmaceutical, therapeutic or medicinal treatment. In the rubbery state, time to degradation would be more rapid due to transport of reactive species within the solid.

The term “premature” as used herein can refer to a human baby born before reaching 37 weeks' gestational age verses normally 40 weeks of gestational age at birth.

Reference will now be made to various embodiments, examples of which are illustrated in the accompanying drawings.

Reference will now be made in detail to certain claims of the disclosed invention, examples of which are illustrated in the accompanying drawings. While the disclosed invention will be described in conjunction with the enumerated claims, it will be understood that they are not intended to limit those claims. On the contrary, the disclosed invention is intended to cover all alternatives, modifications, and equivalents, which can be included within the scope of the disclosed invention as defined by the claims.

The disclosed innovations and claimed inventions provide a lung surfactant composition (LSC) that can be prepared, shipped and administered as a prepared suspension or emulsion. The LSC can be further processed to an amorphous powder prior to shipment by drying methods supra, as known to the skilled artisan. The resulting powder can be sufficient to keep the LSC stable and has not been demonstrated previously. Additionally, the drying of the surfactant mixture in the presence of pharmaceutically-accepted excipients that form high glass temperature (Tg), amorphous powdered solids can provide a useful treatment for RDS and other respiratory diseases. Moreover, being able to reconstitute the amorphous powder into a concentrated suspension enables dosing at lower volumes while delivering a higher concentration of LSC which has not been accomplished prior to Applicants disclosure. See, for example Biotech. Advances 36 (2018) 1185-1193, see p, 1186. It is also noted that use of the disclosed LSC in conjunction with nebulization would be a novel method of administration. In addition, the use of the LSC as a vehicle for the delivery of other pharmacologically active agents such as non-steroidal or steroidal anti-inflammatories, anti-infectives can be novel uses of Applicant's LSC. Therefore, the various novel LSC embodiments disclosed herein have greater potency because Applicant's LSCs therapeutic concentration can be increased.

In various embodiments of the disclosed innovations and claimed inventions disclosed are heat stable protein-lipid concentrate within the disclosed LSC for the treatment of respiratory diseases. The heat stable protein-lipid concentrate can provide localized delivery of LSC therapeutics including, but not limited to, intratracheal instillation, endotracheal instillation, nebulization and a pulmonary delivery device. Additionally, the heat stable protein-lipid concentrate can facilitate the spread and transport of LSC and bioactive therapeutics to the inner recesses of the lung at the gas/liquid interface within the interior of the alveoli sacs for improved effective dosing and so efficacy of treatment.

The present teachings can be implemented using the disclosed LSC having within a solution, an emulsion, individual liposomes, lipid-protein complexes and suspensions both as naturally occurring, recombinant, homologous/analogous, fragment, modified or mimic lung surfactant protein (having an improved structure) and/or a FAK protein and/or or the V12Rac1 protein possibly fused with a domain (e.g. the HIV Transactivator transport domain, TAT) that enables transport into cells in combination with at least one surfactant protein, a phospholipid and combinations thereof and optionally at least one buffer, optionally at least one excipient and optionally at least one bioactive agent.

Alternatively, the corresponding genes that code for these proteins contained within a nucleic acid vector that has tropism for lung epithelial cells can be mixed with the LSC for delivery to the lung. Lung surfactant proteins are identified in a mammal as a surfactant protein (SP) followed by a letter. SP-A, SP-B, SP-C and SP-D are mammalian pulmonary surfactants.

SP-B

SP-B is a component of pulmonary surfactant, 79 amino acids in length and contains 3 intrachain disulfide bond and 1 interchain disulfide to form a homodimer. This hydrophobic protein is associated with alveolar membranes and it appears that in some conformations it is partially penetrates into the membrane. In association with phospholipid bilayers, SP-B aids the transport of phospholipid into and out of alveolar membranes. In combination with membrane phospholipids such as dipalmitoylphosphatidylcholine (DPPC) surface tension at the alveolar surface is reduced during compression and expansion during each breath cycle, in the same manner as surface tension is reduced during compression and expansion of surfactant droplets as detected by dynamic surface tension. SP-B can also organize lipids below the surface of the gas/fluid interface by cutting and pasting lipid-bilayer pieces to form the 3D structure of tubular myelin and SP-B can arrange lipids into lamellar bodies found inside of type II pneumocytes. Lamellar bodies secreted into the fluid lining the interior of alveoli become tubular myelin, necessary for producing pulmonary surfactant.

In one of the various embodiments various embodiments of the disclosed innovations and claimed inventions, SP-B can be modified by substitution at designated residue positions of select amino acids. Amino acids are numbered from the 5′ N-end to the 3′ C-terminus along the amino acid sequence, reading the 5′ end at the left end to the 3′ terminus at the right end. The first seven amino acids of mature SP-B facilitate membrane insertion of SP-B. The sequence of SP-B, analogs and fragments are illustrated in FIG. 1.

There are numerous SP-B analogs, homologs and fragments that can be included in various embodiments of the disclosed LSC being either a full length SP-B protein or a fragment(s) thereof. Table 1 lists ten amino acid residues and possible substitutions that alter SP-B solubility, increase SP-B pl, and remove the potential for SP-B degradation at methionine, tryptophan, aspartic acid and glutamine.

TABLE 1
SP-B AMINO ACID RESIDUE SUBTITUTION SITES

| | Conserved | Phylogenic | Alternative Non- | Benefit of Phylo-, Non- |
|---|---|---|---|---|
| Residue | Amino Acid | Amino Acid | Phylogenic Amino Acid | Phylogenic Substituted |
| Position | Substituions | Substitutions | Substitutions | Amino Acid(s) |
| W9 | | R | Y | Removes oxidation site |
| A13 | | T | S, E | Increases solubility |
| M21 | | V | Any non-oxidizable amino | Removes oxidation site |
| | | | acid including but not | |
| | | | limited to: L, I, A, P | |
| A32 | | G, S | E, D | More soluble |
| H36 | | R | K | Increases protein pI, |
| | | | | increasing membrane |
| | | | | positive charge |
| C48 | | | Any amino acid not | Removes potential for |
| | | | capable of forming a | homodimerization, |
| | | | disulfide bond including | facilitating synthesis |
| | | | but not limited to: T, E, D | |
| S54 | | T | A | Less soluble |
| M65 | | V | Any non-oxidizable amino | Removes oxidation site |
| | | | acid including but not | |
| | | | limited to: L, I, A, P | |
| R72 | | G | | Greater flexibility and, |
| | | | | solubility |
| M79 | | L, S, N, T | Any non-oxidizable amino | More soluble, Removes |
| | | | acid sidechain including | potential oxidation site |
| | | | but not limited to: I, A, Q, P | |

Substituting amino acids within SP-B as disclosed in Table 1 can result in numerous SP-B analogs as illustrated in FIG. 1.

SP-C

SP-C is only 35 amino acid long, assists in the functions of SP-B and is found within lipid structures. Mature SP-C cannot be formed without SP-B which acts in post-translational modification of SP-C. The amino acid sequence of SP-C can be varied, often at the 3′ end of the peptide sequence but has been normally regarded as invariant as discussed in Johansson, Jan. “Structure and properties of surfactant protein C” Biochim. Biophys. Acta (1998) 1408:161-172. Non-limiting examples of SP-C analogs are illustrated in FIG. 1.

In various embodiments of the disclosed innovations and claimed inventions SP-C can be modified at either the N- and/or C-terminus with the attachment/fusion of an anti-inflammatory peptide therapeutic, an anti-infective peptide therapeutic or to affect other pharmacological effects including, but not limited to, endothelial tissue repair in ARDS. Any one of these SP-C modifications can function to inhibit and/or counteract/provide therapeutic efficacy for inflammatory pathology(s) tissue repair, combating microbial and viral infection(s) and recruitment of the innate immune response.

TAT-FAKp

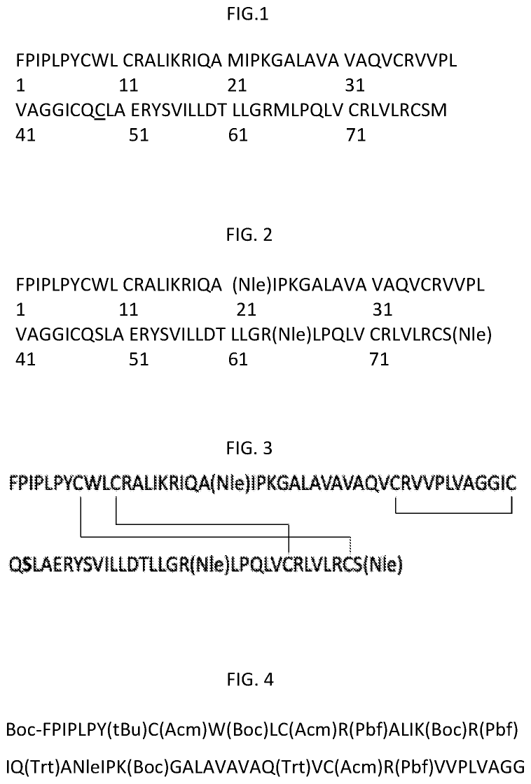

The TAT-FAKp protein alone or in combination with SP-B and/or SP-C within the individual liposomes within the emulsion, suspension or lipid-protein complexes in various embodiments of the disclosed innovations and claimed inventions there can be proposed a way to provide a fast-acting pharmacospecific therapy for acute lung injury (ALI) and ARDS. TAT-FAKp protein combines purified, phosphorylated form of focal adhesion kinase (FAKp), or another molecule in which a peptide or protein is intended for intracellular delivery, with a transport protein, abbreviated as TAT. ACCESSION NO: L05186; VERSION L05186.1 has the complete CDS for human FAKp mRNA and the encoded amino acid sequence. Exemplary TAT proteins suitable for use in the claimed invention include, but are not limited to Chariot©, pepetratin, TAT fragment, a signal sequence-based peptide, and transportan are presented in FIG. 2

FAKp can be administered as a complex with TAT, a cell permeable peptide (CPP). TAT, covalently fused to NTA (nitrilotriacetic acid)) forms a complex with FAKp through a copper cation which forms a TAT→Cu²⁺←F histidine-tagged-FAKp complex. The cDNA for making recombinant FAKp for use in the TAT-FAKp protein complex would be a histidine-tagged cDNA that encodes full-length human FAK that is constitutively phosphorylated at tyrosine 397. The protein or peptide or a biologically active fragment, or variant thereof of FAKp can be noncovalently bound to a TAT protein. The FAKp with the histidine tag, the TAT protein can be chelated to a metal cation selected from Cu, Ni, Zn, and Co. The metal ion can be noncovalently bound to the histidine tag on the FAKp as a reversible bond, thus forming the TAT-FAKp complex.

The presence of TAT-FAKp within the lung endothelium can induce barrier enhancing protein-protein interactions to protect against acute lung injury (ALI) (see U.S. Pat. No. 8,420,080). Protein-protein interactions can be between FAK, paxillin, vinculin and α-actinin-1, forming a protein complex (the FPVA complex). The FPVA complex enhances F-actin at adherens junctions resulting in cadherin clustering and barrier enhancement. It is postulated that the loading of endothelial cells containing TAT-FAKp will abrogate thrombin or oxidant-induced hyperpermeability in vitro in cultured human lung microvascular, pulmonary artery endothelial cells, and in mouse lungs as seen in lung endothelial monolayers and ALI-induced lung hyperpermeability in mice. The lung surfactant complex can also have a buffer and excipient to improve the glass transition (Tg) temperature such that the individual lipid-protein complexes do not aggregate in the LSC in the suspension, emulsion or reconstituted amorphous powder.

CHF5633

CHF5633 is a synthetic reconstituted surfactant protein having SP-B and SP-C analogs and is considered a promising alternative to animal-derived therapeutic preparations for treatment of IRDS. It is composed of DPPC and POPG lipids, sodium salt and a synthetic SP-C analog (IPSSPVHLKRLKLLLLLLLLILLLILGALLLGL, SEQ ID NO:7) and a synthetic SP-B analog (CWLCRALIKRIQALIPKGGRLLPQLVCRLVLRCS, SEQ ID NO:4 as active ingredients. In CHF5633 it is noted that Cys-3 and Cys-4 residues were replaced by Ser-3, Ser-4 in the SP-C analog supra, because the pair of cysteines are not simply cysteine amino acids, but each has a fatty acid side chain (long chain fatty acid as part of the peptide backbone) that can form a thiol ester. The fatty acids each lose an —OH group and combines with sulfur to form a thiol ester. Alternatively, replacement of the fatty acid with an —ROH group can form either a thiol ether or an ether, as thiol esters are unstable. Applicant is unaware of literature suggesting formation of a thiol ether or ether to create a stable analog of SP-C Further details are found in Seehase, M. et al. “New Surfactant with SP-B and C Analogs Gives Survival Benefit after Inactivation in Preterm Lambs” (2012) PLOS One 7(10): e47631. doi:10.1371/journal.pone.0047631 and Sweet, D G, Turner M A, Stranak Z. et al. Arch Dis Child Fetal Neonatal Ed. (2017); 102:F497-F503. (2017) “A first-in-human clinical study of a new SP-B and SP-C enriched synthetic surfactant (CHF5633) in preterm babies with respiratory distress syndrome”.

There are two forms, a mini CHF5633 (MB):

(SEQ ID NO: 4)
CWLCRALIKR IQAMIPKGGR MLPQLVCRLV LRCS

and a 41 amino acid peptide, supermini CHF5633 (S-MB) having SP-B residues 1-7 covalently attached to the N-terminus of MB:

(SEQ ID NO: 9)
FPIPLPYCWL CRALIKRIQA MIDATKRMLP QLVCRLVLRC S

(Notter R H, et al. “Activity and biophysical inhibition resistance of a noel synthetic lung surfactant containing Super-Mini B DATK peptide.” Peer J. 4:31528 (doi.org/10.7717/peerj.1528)).

Among other things, the protein-lipid complex of the LSC can have at least one of a fatty acid, a lipid and a phospholipid collectively referred to herein as “lipid” and possibly combinations thereof. Fatty acids are well known in the art and possible fatty acids can include but are not limited to saturated fatty acids including, but not limited to butyric, caproic, caprylic, capric, lauric, myristic, palmitic, stearic, arachidic, behenic, lignoceric, oleic, ricinoleic, vaccenic, linoleic, α-linolenic, γ-linolenic, gadoleic, arachidonic, eicocsapentaenoic (EPA), erucic, docosahexaenoic acid (DHA), maternal milk, and algae oil as is known to the skilled artisan. Longer chain fatty acids, being more hydrophobic are known to be less soluble while fatty acids with double bonds between carbons can increase solubility, decreased melting temperature with unsaturated fatty acids existing as solids at room temperature (about 25° C.).

Lipids can include, but are not limited to, natural and synthetic phosphoglycerolipids, sphingolipids and digalactosylglycerlipids as is known to the skilled artisan. Further, in various embodiments of the disclosed innovations and claimed inventions, the lipid(s) can be one or more of phosphaltidylcholine (PC), lysophosphatidylcholine (LPC), phosphatidylethanolamine (PE), lysophosphatidylethanolamine (LPE), phosphatidylglycerol (PG), lysophosphatidylglycerol (LPG), phosphatidylinositol (PI), lysophosphatidylinositol (LPI), phosphatidylserine (PS), lysophosphatidylserine (LPS), cholesterol (Choi), palmitoyloleoylphosphatidylglycerol (POPG), dipalmitoylphosphatidylcholine (DPPC), 1,2-Dipalmitoyl-sn-glycero-3-phosphoethanolamine (DPPE), 1,2-dipalmitoyl-sn-glycero-3-phosphoglycerol (DPPG), dioleoyl phosphatidylcholine, (DOPC), 1,2-Dioleoyl-sn-glycero-3-phosphocholine, palmitoyloleoylphosphatidylcholine (POPC), 1-Palmitoyl-2-oleoyl-sn-glycero-3-phosphoethanolamine (POPE), 1-palmitoyl-2-oleoyl sn-glycero phosphocholine (POPS), 1,2-Distearoyl-sn-glycero-3-phosphocholine (DSPC), palmitoleic, oleic, ricinoleic, vaccenic, linoleic, α-linolenic, γ-linolenic, gadoleic, arachidonic, eicocsapentaenoic (EPA), erucic, docosahexaenoic acid (DHA) sphingomyelin (SM), sphingosine, ceramide, cerebroside, ganglioside, SM with an amine, SM with a quaternary amine, SM that incorporates phosphocholine, SM that includes a phosphoethanolamine, dilauroylphosphatidylcholine (DLPC), disteroylphosphatidylcholine (DSPC), behenoylphosphatidyl-choline, arachidoylphosphatidylcholine (ADPC), [(+)-trimethyl(3-phosphonopropyl)ammonium, mono(2-hexadec-9-enyloxy-3-hexadecyloxypropyl) ester] (DEPN-8), diether phosphono-phosphatidylglycerol (PG-1), and combinations and modified versions thereof.

DEPN-8 is a phospholipase-resistant phospho-choline derivative and PG-1 is a phospholipase-resistant C16:0, C16:1 diether phosphono-phosphatidylglycerol where the phosphatidyl group is attached to the C1 carbon of glycerol (disclosed in U.S. Pat. No. 9,815,869).

In various embodiments of the disclosed innovations and claimed inventions the composition can further comprise a buffer including, but not limited to sodium, potassium, calcium and/or lithium buffers selected from the group including acetic acid, phosphoric acid, citric acid, boric acid, histidine, lactic acid, tromethamine, gluconic acid, aspartic acid, glutamic acid, tartaric acid, succinic acid, malic acid, fumaric acid, and alpha-ketoglutaric acid. The sodium, potassium, calcium and/or lithium buffer can have the sodium, potassium, calcium and/or lithium salt with the conjugate base or the salt's conjugate acid. The selected buffer would be known to the skilled artisan to be suitable in spray-drying applications of the disclosed compositions.

A less diluted LSC amorphous powder could be more suitable for NRDS while a more concentrated LSC could be more advantageous in the treatment of ARDS. Alternatively, the amorphous powder can be reconstituted for administration at a higher concentration in a smaller delivery volume to achieve greater efficacy and efficiency of treatment. The higher concentration can have a delivery volume of less than 1.25 ml/kg and up to about 5.0 ml/kg. The volume can be about 1.0, 1.05, 1.1, 1.15, 1.2, 1.24, 1.25, 1.5, 1.75, 2.0, 2.25, 2.5, 2.75, 3.0, 3.25, 3.5, 3.75, 4.0, 4.25, 4.5, 4.75, 5.0 5.25 and 5.50 ml/kg. For an adult ARDS patient the dose can be up to 40.0-500.0 mg/kg. The doses can be about 25.0, 30.0, 35.0, 40.0, 45.0, 50.0, 55.0, 60.0, 65.0, 70.0, 75.0, 80.0, 85.0, 90.0, 95.0 100.0. 105.0, 110.0, 115.0 and 120.0, 130.0, 140.0, 150.0, 160.0, 170.0, 180.0, 190.0, 200.0, 220.0, 240.0, 260.0, 280.0, 300.0, 325.0, 350.0, 375.0, 400.0, 425.0, 450.0, 475.0, and 500.0 mg/kg.

In addition, there can also be excipients selected from the group consisting of one or more of polyol(s) protein(s), sugar(s). Polyols can be selected from the group consisting of one or more of erythritol, inulin, lactitol, maltitol, mannitol, myoinositol, sorbitol, xylitol and hydrates thereof. Sugars can include, but are not limited to, natural and synthetic sugar(s) including, but not limited to, dextran, fructose, galactose, glucose, inulin, maltose, mannitol, raffinose, melezitose, sorbitol, stachyose, sucrose, trehalose, starch and hydrates thereof. The amino acids can include, but are not limited to proteins having amino acid polymers such as phenylalanine, cystine, glycine, arginine, histidine, lysine, and leucine. The amino acid excipients can be selected from the group consisting of one or more of L-phenylalanine, L-cystine, glycine, L-arginine, L-histidine, L-isoleucine, L-lysine, and L-leucine, L-proline, L-methionine, L-threonine, L-tryptophan, L-valine, L-glutamic acid, L-aspartic acid, L-asparagine, L-glutamine, L-tyrosine, L-serine, L-alanine, tri-leucine and salts thereof.

In various embodiments of the disclosed innovations and claimed inventions the lung surfactant can be dried to form an amorphous powder. The reconstitution of the amorphous powder into at least one of an emulsion and a suspension can significantly increase the dosing concentration of the LSC per unit volume. Previous attempts to increase the overall dose by increasing only the number of vials administered has not resulted in efficacy or efficient per kg body weight as seen in the treatment of IRDS. Thus, providing greater concentrations in mg/ml of deliverable lung surfactant in a concentrated dose exceeding up to 200 to 500 mg/kg to 600 mg/kg dosed can have the potential to improve ARDS treatment outcomes (See Kim and Won, (2018) Biotech. Adv.36:1185-1193).

Selection of an appropriate excipient with or without buffer in various embodiments for making the disclosed LSC can facilitate formation of an emulsion or suspension that following drying into a powder results in formation of an amorphous powder. The lung surfactant can be dried to an amorphous powder by at least one of spray drying, supercritical fluid freezing, bubble drying, and lyophilization as known to the skilled artisan. Knowing the stating concentration of the surfactant protein(s) and lipid(s), the skilled artisan can readily determine the concentration of the reconstituted lung surfactant by how much reconstituting liquid, buffer, dilute organic solvent and combinations thereof can be used.

In various embodiments of the disclosed innovations and claimed inventions the formation of LSC as a stable, amorphous powder can permit shipping and storing of the LSC as an amorphous powder without the need for refrigeration. Excipients that form amorphous powders are preferred since crystalline powders form a crystalline solid phase that phase separates from the lipid-protein complexes. Amorphous powders can more easily separate into individual liposomes or protein-lipid complexes. Excipients that form amorphous powders with particularly high glass transition temperatures (Tg >50 deg C. or more) can be selected as they will retain rigidity and separation of the liposomes or lipid-protein complexes even at elevated temperatures.

The resulting amorphous powdered LSC can have a glass transition temperature (Tg) of a least 50° C., 55° C., 60° C., 65° C., 70° C., 75° C., 80° C., 85° C., 90° C., 95° C., 100° C., 105° C., 110° C., 115° C., 120° C., 125° C., 130° C., 135° C., 140° C., 145° C., 150° C., 160° C., 170° C. to 180° C. The Tg can be about 50-180° C., 70-150° C., 50-130° C., 60-130° C., 70-140° C., 60-160° C., 55-170° C., 50-150° C., and 50-140° C.

The resulting amorphous powdered LSC can remain stable, including, but not limited to, free from degradation, decomposition and loss of efficacy and/or potency at temperatures of a least 40° C., 45° C., 50° C., 55° C., 60° C., 65° C., 70° C., 75° C., 80° C., 85° C., 90° C., 95° C., 100° C., 105° C., 110° C., 115° C., 120° C., 125° C., 130° C., 135° C., 140° C., 145° C., 150° C., 160° C., 170° C. to 180° C. The amorphous powder can be stable from about 40-180° C., 45-170° C., 50-160° C., 40-140° C., 40-135° C., 40-130° C., 50-180° C., 70-150° C., 50-150° C., 60-140° C., 70-140° C., 60-160° C., 55-150° C., 50-150° C., 45-150° C., 45-145° C., 45-140° C., 50-140° C., and 40-150° C.

Suitable sterile liquids for reconstitution of lung surfactant from an amorphous powder, in various embodiments of the disclosed innovations and claimed inventions, lung surfactant can include, but are not limited to water, distilled water, reverse osmosis (RO) water, isotonic saline, buffer solution, a dilute organic solvent and combinations thereof. In various embodiments of the claimed composition, the aqueous diluted organic solvent can be at a concentration of at least: 1.5%, 2.0%, 2.5%, 3.0%, 3.5%, 4.0%, 4.5%, 5.0%, 5.5%, 6.0%, 6.5\hat{ }, 7.0%, 7.5%, 8.0%, 8.5%, 9.0%, 9.5% and up to 10.0%. Organic solvents, including dimethyl sulfoxide (DMSO) and dimethylformamide (DMF) can be used at a concentration of less than 5% and ethanol can be used at a concentration of up to 10% when reconstituting the amorphous powder.

Respiratory diseases, including but not limited to, Adult respiratory distress syndrome (ARDS), IRDS, BPD, pulmonary injury can have accompanying infections that can be adverse to treatment efficacy and efficiency. In various embodiments of the disclosed innovations and claimed inventions the LSC compositions can also include at least one bioactive agent. The bioactive agent can be one or more anti-inflammatory and/or anti-infective agents including, but not limited to, anti-inflammatory therapeutics, anti-infective drugs, biologics, bronchodilation drugs, antihistamines, cyclooxygenase inhibitors, leukotriene antagonists, PLA2 inhibitors, PAF antagonists, analgesics, leukotriene inhibitors or antagonists, decongestants and anti-tussive drug substances, anticholinergics, β-blockers, β₂-adrenogenic receptor agonist, anesthetics, anti-tuberculars, cardiovascular agents, and combinations thereof (disclosed in U.S. Pat. No. 9,050,267).

In various embodiments of the disclosed innovations and claimed inventions bioactive agents can include, but are not limited to, imaging agents, enzymes, steroids, genetic material, nucleic acid vectors, antisense agents, nucleic acid aptamers, mesenchymal stem cells, CAR-T cells, biologics, proteins, peptides and combinations thereof (disclosed in U.S. Pat. No. 9,050,267).

In various embodiments of the disclosed innovations and claimed inventions bioactive agents can include, but are not limited to, anti-inflammatory agents including, but not limited to omega-3 poly-unsaturated fatty acids (PUFA) including eicocsapentaenoic (EPA), docosahexaenoic acid (DHA), flunisolide, budesonide, tripedane, cortisone, fluticasone (e.g. propionate), mometasone (e.g. furoate), dexamethasone, beclomethasone, betamethasone, and triamcinolone (e.g. acetonide), adrenaline (ephedrine), fenoterol, formoterol, isoprenaline, metaproterenol, mometasone, prednisone, prednisolone, methyl prednisolone, and triamcinolone. Anti-inflammatory agents can also include nonsteroidal anti-inflammatory drugs (NSAIDs) including, but not limited to, one or more of aspirin, naproxen, acetaminophen, diclofenac, celecoxib, ibuprofen, budesonide, butixocort tixocortol butyrate), diflunisal, indomethacin, etodolac, ketoprofen, ketorolac, nabumetone, oxaprozin, piroxicam, salsalate, sulindac, and tolmetin.

In various embodiments of the disclosed innovations and claimed inventions the anti-inflammatory agent is a β₂-adrenoreceptor agonist (bronchodilator) including, but not limited to, one or more of: albuterol (aka salbutamol), bitolterol, fenoterol, iosprenaline, levosalbutamol, orciprenaline, pirbuterol, proaterol, ritodrine, salbutamol, terbutaline, arformoterol, bambuterol, clenbuterol, formoterol, salmeterol, abediterol, carmoterol, indacaterol, olodaterol, vilanterol, isoxsuprine, mabuterol, and zilpaterol.

Anti-infective therapeutic agents can, in various embodiments of the disclosed innovations and claimed inventions, be included in the LSC such as antibiotics, antibacterials, antifungals, antivirals, anti-protozoans, and peptides.

Antimicrobial peptides (AMPs) as anti-infective bioactive agents are known to have activity against bacteria, viruses, fungi and unicellular protozoa and can be components of innate immunity to protect the host against infections and are active in pathogen clearance. Reviews of AMPs can be found in Mahlaupuu, Margit et al., (2016) Front. Cell. Infect. Microbiol. 27 Dec. 2016 (doi.org/10.3389/fcimb.2016.00194).

Peptides, used as small molecule therapeutic agents against disease having an inflammatory component can include, but are not limited to, one or more of CATH-1, CATH-2, LL-37 and CRAMP. A review of therapeutic peptides can be found in La Manna, Sara et al. “Peptides as Therapeutic Agents for Inflammatory-Related Diseases” Int. J. Mol. Sci. (2018) 19(9)2714; doi.org/10.3390/ijms19092714.

Method of Making

In various embodiments of the disclosed innovations and claimed inventions the claimed LSC can be made by forming a protein-lipid complex having at least one lung surfactant protein and homologs, analogs, fragments and mimics thereof. The SP can be SP-A, SP-B, SP-C, and SP-D, a combination of SP-B+SP-C, and SP-C+TAT-FAKp and FAKp attached to SP-C. Optionally, within the composition can also be at least one buffer and/or at least one excipient and at least one bioactive agent.

The ratio of SP(s) to lipid(s) can be adjusted and the selection of lipid(s) can be varied to facilitate ease of spreading of the LSC within the alveoli. Naturally occurring LSC is about 90% lipid and 10% protein. However, selection of lipid and protein sequence optimization to facilitate penetration of the lipid(s) into the alveoli's lipid bi-layer can both facilitate gas exchanges occurs by adsorbing oxygen at the air-water interface of alveoli and with the addition of an anti-inflammatory and/or anti-infective, preclude degradation of SP.

Ratios of percent lipid to percent protein in the LSC can be from 8.0:0.10, 8.0:0.25, 8.0:05.0, 8.0:0.75, 8.0:1.0, 8.0:1.25, 8.0:1.50, 8.0:1.75, 9.0:0.10, 9.0:0.25, 9.0:05.0, 9.0:0.75, 9.0:1.0, 9.0:1.25, 9.0:1.50, 9.0:1.75, 10.0:0.10, 10.0:0.25, 10.0:05.0, 10.0:0.75, 10.0:1.0, 10.0:1.25, 10.0:1.50, 10.0:1.75, 11.0:0.10, 11.0:0.25, 11.0:05.0, 11.0:0.75, 11.0:1.0, 11.0:1.25, 11.0:1.50, 11.0:1.75, 12.0:0.10, 12.0:0.25, 12.0:05.0, 12.0:0.75, 12.0:1.0, 12.0:1.25, 12.0:1.50, 12.0:1.75, 13.0:0.10.

DPPC in conjunction with another lipid(s) supra, improves uniformity of coverage as well as the LSO's ability to penetrate into the deep recesses of the lung's alveoli. Drying the disclosed LSC to an amorphous powder which can be resuspended prior to use can restore the ability of the LSC to spread over the lung and alveoli surfaces without the loss of the ability to alter surface tension and for transferring of lipid(s) between lipid bilayers and other lipid structures.

The lung surfactant complex components are mixed, forming an emulsion and/or a suspension. At least one bioactive agent supra, can be added either prior to or after formation of the emulsion and/or suspension. The addition of the bioactive agent increases the total solids within the LSC and the concentration of bioactive agent can be a consideration along with the age, weight and overall health of the mammal to which the LSC would be administered for consideration of dosing concentration in mg/kg and well as dosing volume in ml/kg.

Selection of buffer and/or excipient can also increase the total solids within the LSC and the concentration of each would also be taken into consideration when drying the LSC into an amorphous powder as discussed supra.

Reconstitution of the amorphous powder can permit the dosing concentration of LSC to be adjustable based on the age, weight and overall health of the mammal in need of treatment for a respiratory disease. Liquids and solutions suitable for use in reconstitution are discussed supra. The mammal can be any one of a premature infant, infant, child, and adult.

Treatment of Respiratory Diseases

Respiratory diseases broadly refer to conditions which make gas exchange an obstacle in air-breathing animals. Most often there is an underlying pathology within the respiratory system's organs, e.g., lungs, bronchi, pharynx, larynx, and diaphragm. Conditions of the respiratory tract can include the trachea, bronchi, bronchioles, alveoli, pleurae, pleural cavity as well as the nerves and muscles involved in respiration—the inhalation and exhalation of air and carbon dioxide, respectively. The alveoli make up the respiratory surface where gas exchange occurs by adsorbing oxygen at the air-water interface of alveoli. They are composed of microscopic air sacs that exchange oxygen at the liquid/gas interface on the interior surface of the alveoli with circulating blood which cares inhaled oxygen throughout the body and returns and exchanges carbon dioxide at the water/air interface into the alveoli, back into the lungs for exhalation. Respiratory diseases can be a consequence of blockage within the airway, an obstructive lung disease. A restrictive lung disease results from incomplete lung expansion and lung stiffness as seen in IRDS. This can be associated with or resulting from deficient and/or dysfunctional lung surfactant production by the patient in need of treatment.

Respiratory diseases associated with deficient lung surfactant include, but are not limited to, infant/neonatal respiratory distress syndrome (IRDS/NRDS), adult respiratory distress syndrome (ARDS), bronchopulmonary dysplasia (BPD), pulmonary injury, and infection leading to late onset sepsis. Both infant and adult respiratory distress syndromes can result from and include, but are not limited to acute lung injury (ALI), ventilator-induced lung injury (VILI), and systemic inflammatory response syndrome (SIRS). Moreover, ARDS can result from one or more causes including, but not limited to, shock, bacterial, viral and nosocomial pneumonias, and inhalation of toxic gases, vapors, fumes and particles. Other respiratory diseases for which various embodiments of the claimed invention can be useful in treating respiratory diseases include, but are not limited to, asthma, cystic fibrosis (CF), chronic obstructive pulmonary disease (COPD), and Lysosomal storage diseases. Complications of SARS COVID-19 may also benefit from the disclosed composition for use within a combination treatment therapeutic composition or for delivery of therapeutics alone or in combination.

Pulmonary injury can be a consequence of bronco-biopsy, mechanical ventilation, bronchopulmonary dysplasia, late onset sepsis, administration of oxygen, aspiration, inhalation of toxic gases, vapors, fumes and particles, and intubation.

It is noted that infection can be a consequence of bacterial, viral, protozoan, and nosocomial infection sources. Pulmonary injury can also occur due to pancreatitis, transfusion associated acute lung injury (TRALI), drug overdose with various agents, near drowning (inhalation of fresh or salt water), hemmorhagic shock or reperfusion injury, smoke inhalation, as well as aspiration of gastric contents into the lungs, poor sanitation, an infected wound, and inadvertent and/or accidental inhalation of chemical vapors, particulates, smoke and fumes. A detailed description of respiratory infections and diseases can be found in Dasaraju and Liu, Infections of the Respiratory System. In: Medical Microbiology. 4th ed. S. Baron et al., Co-Editor, 1996, U. Texas Med. Branch, Galveston, TX. ISBN 0963117211 and in Matthay, M. A. et al., “Acute respiratory distress syndrome, Nat. Rev. Disease Primers 5, Article No. 18 (2019) https://doi.org/10.1038/s41572-019-0069-0, each of which are incorporated by reference herein.

Injury of the microvasculature resulting from any of the causative factors described leads to leakage of white and red blood cells, platelets, clotting factors and other components in blood circulation. Therefore, the microvasculature of any organ will be adversely affected when the integrity of the endothelial cells that make up these tissues is loosened. This can be countered by compounds that lead to upregulation of VE-cadherin on the surface of these cells by compounds such as focal adhesion kinase (FAK), angiopoietin-1, small GTPases, intracellular modulators, catenins, plakoglobin and VE-protein tyrosine phosphatase. In addition to acute respiratory distress syndrome, the resulting morbidities include microvascular coronary disease (stroke and myocardial infarction), acute and ventilator-induced lung injury, sepsis, pancreatitis, cerebral small-vessel disease, preeclampsia, pulmonary arterial hypertension (PAH), endothelial dysfunction in diabetes, diabetic cardiomyopathy, rheumatoid arthritis, systemic lupus erythematosus, asthma, neoplasms, diabetic retinopathy or age-related macular degeneration, and systemic sclerosis, may have a common etiologic linkage related to microvascular disease.

In addition, a Systemic Capillary Leak Syndrome, characterized by arterial hypotension, hemoconcentration and low albumin levels with hypotensive shock and anasarca. In these patients, pro-inflammatory and endothelial mediators are significantly increased and prompt treatment is required.

Administration of the LSC, in various embodiments of the disclosed innovations and claimed inventions, can be by inhalation, pharyngeal aspiration, intratracheal instillation, endotracheal instillation, nebulization and a pulmonary delivery device. Depending on the administration route used, the LSC can be in the form of an aqueous emulsion, an aqueous suspension, an amorphous powder (AP), a reconstituted AP in an aqueous suspension having a pharmaceutically acceptable carrier, and a reconstituted AP in a dilute organic suspension comprising a pharmaceutically acceptable carrier. At least one bioactive agent can be either in solution or as part of the liposome, emulsion of the LSC, and as a fusion protein to the SP within the LSC for simultaneous administration. Alternatively, the bioactive agent can be administered, sequentially, separately in various embodiments of the disclosed innovations and claimed inventions.

Suitable pulmonary delivery devices can include but are not limited to nebulizer, inhaler, direct intubation in the lung of an infant and adding the LSC with a syringe drop-wise or by squirting using the INSURE technique: INtubate, SURfactant, Extubate.

The format and delivery of the disclosed LSC can provide an economical solution for treating respiratory diseases. The disclosed LCS can be an affordable therapeutic that i) does not require a high level of expertise to administer, ii) can be easily stored in the lypholized form which needs no refrigeration, and iii) can provide a treatment currently elusive in economically disadvantaged countries throughout the world.

Kits

In another aspect, disclosed is a kit for treating a mammal suffering from a lung surfactant deficiency or having a defective lung surfactant component. In various embodiments of the disclosed innovations and claimed inventions, the kit can have one or more vials of LSC in suspension or emulsion, one or more vials of LSC in an amorphous powdered form. The kit can also have at least one of a sterile reconstitution liquid, a vial(s) of one or more bioactive agent(s), inter-tracheal and/or inter-bronchial catheter(s), syringe(s), sterile wipes, sterile filter(s) for producing sterile dilution and/or reconstitution liquid, and sterile vials.

Any of the compositions described herein can be included in a kit. This kit can be used to practice the invention by providing the therapeutic and delivery materials for treating a mammal suffering from including, but not limited to, IRDS, ARDS, BPD and the like. In a non-limiting example, the kit in suitable container means, comprises one or more of: an LSC in liquid form, LSC in an amorphous powdered form, a delivery mechanism for administering LSC. The kit can further contain one or more sterile: excipient(s), buffer(s), bioactive agent(s), inter-tracheal and/or inter-bronchial catheter(s), syringe(s), needle(s) for e.g., extracting LSC liquid and/or a reconstitution liquid, and/or a bioactive agent, sterile wipes, filter(s) for producing sterile dilution or reconstitution liquid. Optionally, the kit can further contain sterile vials and instructions for use.

The containers of the kits can generally include at least one sterile vial, bottle, syringe or other containers, into which a component can be placed, and preferably, suitably aliquoted. Where there is more than one component in the kit, the kit also can generally contain a second, third or other additional sterile containers into which the additional components can be separately placed. However, various combinations of components can be comprised in a container.

When the components of the kit are provided in one or more liquid solutions, the liquid solution can be an aqueous solution, a suitable sterile aqueous liquid, an LSC suspension, an LSC emulsion. However, the components of the kit can be provided as dried amorphous powder. When reagents and/or components are provided as a dry amorphous powder, the powdered form can be reconstituted by the addition of a suitable sterile dilute organic solvent, a sterile aqueous liquid. The powder can also be reconstituted by the addition of a suitable sterile aqueous solution.

A kit can include instructions for employing the kit components as well the use of any other reagents not included in the kit. Instructions can include variations that can be implemented.

Abbreviations:

| | |
|---|---|
| A = L-alanine | M = L-methionine |
| C = L-cysteine | N = L-asparagine |
| D = L-aspartic acid/L-aspartate | Nle = L-norleucine |
| E = L-glutamic acid/L-glutamate | P = L-proline |
| F = L-phenylalanine | Q = L-glutamine |
| G = glycine | R = L-arginine |
| H = L-histidine | S = L-serine |
| I = L-isoleucine | T = L-threonine |
| L = L-leucine | Y = L-tyrosine |
| V = L-valine | T = L-threonine |
| W = L-tryptophan | |
| Acm = acetamidomethyl, | |
| ACN = acetonitrile | |
| Boc = t-butyloxycarbonyl | |
| DCM—dichloromethane | |
| DIPEA = diisopropylethylamine | |
| DMF = N,N′-dimethylformamide | |
| DPPC = dipalmitoylphosphatidyl choline | |
| EDT = 1,2-ethanedithiol | |
| Fmoc = fluorenylmethoxycarbonyl | |
| Gn•HCl = guanidine hydrochloride | |
| HATU = Hexafluorophosphate | |
| Azabenzotriazole Tetramethyl Uronium | |
| HBTU = (2-(1H-benzotriazol-1-yl)-1,1,3,3- | |
| tetramethyluronium hexafluorophosphate | |
| LCMS = liquid chromatography monitored | |
| by mass spectrometry | |
| MeOH = methanol | |
| MPAA = 4-mercaptophenylacetic acid | |
| Pbf = 2,2,4,6,7-pentamethyldihydrobenzofuran- | |
| 5-sulfonyl- | |
| PG = phosphoglycerol | |
| POPC = 1-Palmitoyl-2-oleoyl-sn-glycero-3- | |
| phosphocholine | |
| TCEP = tris(2-carboxyethyl)phosphine | |
| TFA = trifluoroacetic acid | |
| Trt = triyl | |

### EXAMPLES

The disclosed LSO's can be described in more detail by way of Examples. However, the present invention is not limited to these Examples.

### Example 1 Peptide Synthesis of Fragment 2425: [46-79, C48S, M71Nle, M79Nle]-SP-B

The synthesis of Monomeric SP-B peptide is based on the parent sequence of SP-B (SEQ ID NO:1, FIG. 1). The synthetic sequence of Monomeric-SP-B [M21Nle,C48S,M65Nle,M79Nle] is illustrated in FIG. 2 (SEQ ID NO:2). FIG. 3 depicts the structure of Monomeric SP-B. The solid lines indicate di-sulfide bonds formed within the peptide.

Preparation of the Peptide Resin for Synthesis of SP-B Fragment 2425:

CQSLAERYSVILLDTLLGRNIeLPQLVCRLVLRCSNIe (SEQ ID NO:11).

2-chlorotrityl chloride resin (Cat #12996; Chemlmpex, Wood Dale, IL, 60191) 20.0 g, 6.0 mmol) was added to a glass reaction vessel. The resin was washed with DCM (2×400 mL), swelled in 400 mL DCM for 10 min and drained. Fmoc-Nle-OH (3.4 g, 9.6 mmol) was weighed into this reaction vessel and dissolved in DCM (400 mL) and added into the reaction vessel above. Then DIPEA (6 ml, 36 mmol) was added dropwise. After that, the mixture was shaken at room temperature for 2 hr. The mixture was then drained and DCM/MeOH/DIPEA (400 mL, v/v/v=85:10:5) was added and shaken for 30 min. The DCM/MeOH/DIPEA addition step was repeated.

After draining, the resin was washed with DMF (6×400 mL). Then the resin was washed with MeOH (2×400 mL) and diethyl ether (2×400 mL). After that, the resin was dried under vacuum for 2 hr. Fmoc Loading test: peptide resin: 23.2 g, 6 mmol, loading: 0.223 mmol/g.

A portion of the Fmoc-Nle loaded 2-chlorotrityl chloride resin (9.0 g, 2.0 mmol) was washed with DMF (200 mL) and then drained completely. Fmoc was removed by treatment of resin with 20% piperidine/DMF (200 mL) with 15 mins, two times. The resin was then washed with DMF (6×200 mL).

Assembly of SP-B Fragment 2425 Peptide Chain

Each of the Fmoc amino acids (obtained from GL Biochem Ltd. Minhang District, Shanghai, China) were prepared individually in DMF (200 mL): Fmoc-amino acid Fmoc-Ser(tBu)-OH (2.3 g, 6.0 mmol), Fmoc-Cys(Acm)-OH (2.48 g, 6.0 mmol), Fmoc-Arg(Pbf)-OH (3.89 g, 6.0 mmol), Fmoc-Leu-OH (2.12 g, 6.0 mmol), Fmoc-Val-OH (2.04 g, 6.0 mmol), Fmoc-Leu-OH (2.12 g, 6.0 mmol), Fmoc-Arg(Pbf)-OH (3.89 g, 6.0 mmol), Fmoc-Cys(Trt)-OH (3.51 g, 6.0 mmol), Fmoc-Val-OH (2.04 g, 6.0 mmol), Fmoc-Leu-OH (2.12 g, 6.0 mmol), Fmoc-Gln(Trt)-OH (3.66 g, 6.0 mmol), Fmoc-Pro-OH (2.02 g, 6.0 mmol), Fmoc-Leu-OH (2.12 g, 6.0 mmol), Fmoc-Nle-OH (2.12 g, 6.0 mmol), Fmoc-Arg(Pbf)-OH (3.89 g, 6.0 mmol), Fmoc-Gly-OH (1.78 g, 6.0 mmol), Fmoc-Leu-OH (2.12 g, 6.0 mmol), Fmoc-Leu-OH (2.12 g, 6.0 mmol), Fmoc-Thr(tBu)-OH (2.39 g, 6.0 mmol), Fmoc-Asp(OtBu)-OH (2.47 g, 6.0 mmol), Fmoc-Leu-OH (2.12 g, 6.0 mmol), Fmoc-Leu-OH (2.12 g, 6.0 mmol), Fmoc-Ile-OH (2.12 g, 6.0 mmol), Fmoc-Val-OH (2.04 g, 6.0 mmol), Fmoc-Ser(tBu)-OH (2.3 g, 6.0 mmol), Fmoc-Tyr(tBu)-OH (2.76 g, 6.0 mmol), Fmoc-Arg(Pbf)-OH (3.89 g, 6.0 mmol), Fmoc-Glu(OtBu)-OH (3.66 g, 6.0 mmol), Fmoc-Ala-OH (1.87 g, 6.0 mmol), Fmoc-Leu-OH (2.12 g, 6.0 mmol), Fmoc-Ser(tBu)-OH (2.3 g, 6.0 mmol), Fmoc-Gln(Trt)-OH (3.66 g, 6.0 mmol), Fmoc-Cys(Trt)-OH (3.51 g, 6.0 mmol).

Reagent a: HBTU (2.27 g, 6.0 mmol); Reagent b: DIPEA (2 mL, 12.0 mmol).

Each Fmoc-amino acid (a) was combined sequentially with reagents a, b and the resin. This mixture was mixed and shaken for a minimum of 1.5 hr. The reaction vessel was drained and resin was washed with DMF (6×200 mL).

Coupling efficiency was determined using the ninhydrin test:
i. If the ninhydrin solution was negative (colorless), the next Fmoc amino acid was added as described above.
ii. If the ninhydrin solution turned positive (blue), the same Fmoc-amino acid was re-coupled as described above. Coupling time was increased, if necessary.

Following addition of the last Fmoc-amino acid the resin was washed with DMF (6×200 mL), MeOH (2×200 mL) and diethyl ether (2×200 mL), and the peptidyl-resin was dried (22.5 g, 2.0 mmol). FIG. 4 depicts the protected, synthesized Monomeric SP-B peptide (SEQ ID NO:2) prior to being cleaved from the resin and removing protecting groups.

Cleavage of Peptide from Resin and Removal of Sidechain Protecting Groups

The peptide was cleaved from the resin and sidechain protecting groups were removed by adding the peptidyl-resin (22.5 g, 2 mmol) mixture to a flask and then adding 250 mL of cold cleavage solution (TFA/EDT/thioanisole/H2O=90%/5%/3%/2%, v/v/v/v) and the mixture was reacted for 2 hours at room temperature. The resin was then filtered and washed twice with TFA (˜10 mL each wash). The filtrates were combined and a 10-fold volume of cold diethyl ether was added to the flask. The precipitated peptide was centrifuged and washed with cold diethyl ether three times. The peptide was allowed to dry under reduced pressure to obtain crude peptide (5.9 g). The cleavage reaction of the fragment 2425 peptide chain is illustrated in FIG. 5.

Purification of Fragment 2425

The crude peptide (5.9 g) was purified by prep-HPLC (Mobile Phase: A: 0.05% TFA in water, B: 0.05% TFA in ACN). To an HPLC column (Column: Welch Topsil C18, 21.2×250 mm, 5 μm, 150 Å; Welch Materials, Inc., West Haven, CT.; Catalog No. 00410-01016) 5.9 g of crude 2425 peptide in 300 mL of 50% ACN in water was loaded into the column. The mobile phases were: A: 0.05% TFA in water; and B: 0.05% TFA in ACN. The column had a gradient of 5% B for 3 min, followed by 20-80% B within 20 min, 80-95% B within 0.5 min., and 95% B for 5.5 min. Flow Rate: 25 mL/min.

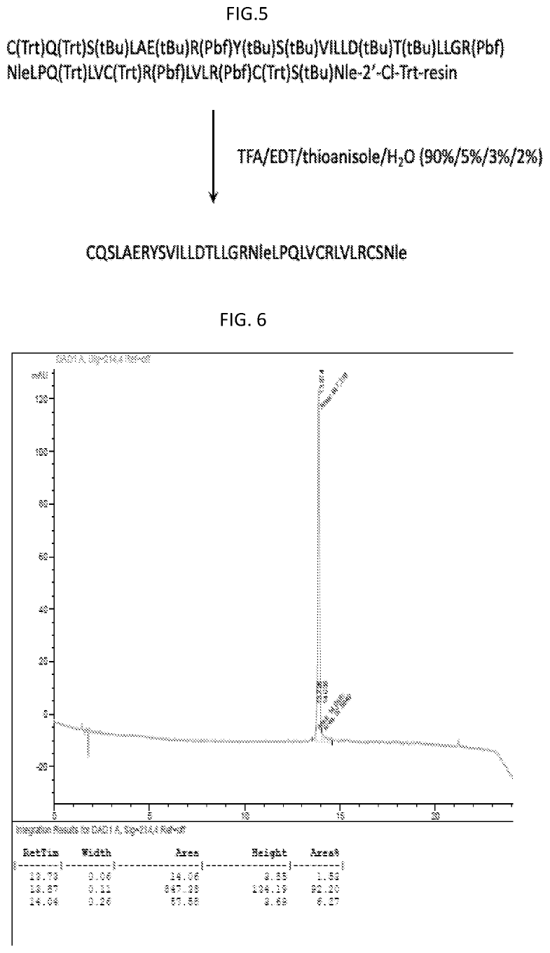

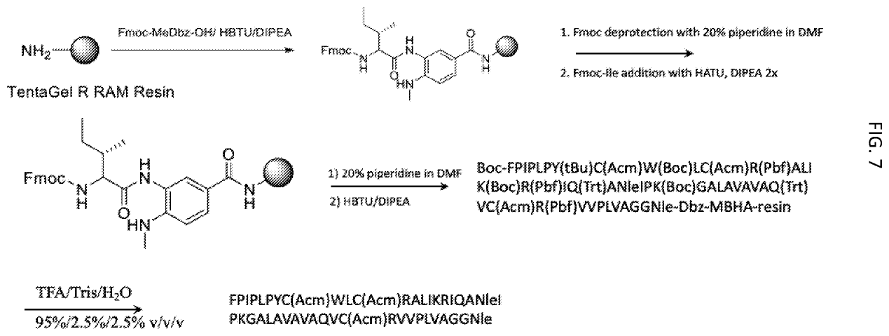

The HPLC profile of SP-B Fragment 2425 material is shown in FIG. 6. The Structure was verified by mass spectrometry. 400 mg of white solid as a TFA salt is obtained (overall yield: 6.7%). X-axis indicates time after sample injection in minutes. Effluent was monitored by UV Spectrophotometry (214 nm). A single quadrupole mass spectrometer was used to verify the mass of Fragment 2425.

LC-MS (ESI) m/z: 1310.3 [M+3H]/3⁺, 983.3 [M+4H]/4⁺, 786.7 [M+5H]/5⁺.

HPLC: RT: 13.78, purity 92.65% (214 nm).

### Example 2 Peptide Synthesis of Fragment 212223 [1-45,8,11,46-Cys(Acm), C-terminal-MeDbz]-SP-B

The synthesis scheme for Fragment 212223 is illustrated in FIG. 7 and entails:

Preparation of the Peptide Resin for Synthesis of SP-B Fragment 212223

(SEQ ID NO: 2)
FPIPLPYC(Acm)WLC(Acm)RALIKRIQANIeIPKGA
LAVAVAQVC(Acm)-RVVPLVAGGI-CO-MeDbz-NH₂

The resin (MeDbz Novasyn® TGR resin, Cat #855157, Millipore Sigma, Taufkirchen, EschenstrafBe 5, Germany; 10.0 g, 2.0 mmol) was washed with DMF (200 mL) and then drained completely. The resin was deprotected by treatment twice with 20% piperidine/DMF (200 mL) for 15 mins. The deprotected resin was washed with DMF (6×200 mL). The resin was modified with Fmoc-MeDbz-OH by adding Fmoc-MeDbz-OH(1.16 g, 3 mmol) and HBTU (1.14 g, 3 mmol) to the resin in DMF (200 ml), than adding DIPEA (1 mL, 3 mmol) and shaken for 1.5 hr. The reaction vessel was drained and the resin was washed with DMF (6×200 mL).

To determine if modification was complete, the ninhydrin test was used as described supra:
i. If negative (colorless), the synthesis was continued.
ii. If positive (blue), FMOC-MeDbz-OH was re-coupled. Increase the coupling time if necessary.

Fmoc was removed from resin by treating two times with 200 mL 20% piperidine/DMF 15 mins. The resin was washed with DMF (6×200 mL).

Coupling Fmoc-Nle-OH to the modified resin. Fmoc-Nle-OH (4.24 g, 12 mmol) and HATU (4.55 g, 12 mmol) were added to the resin in DMF (200 mL), DIPEA (4 mL, 12 mmol) was added, and the mixture shaken for 1.5 hr. The reaction vessel was drained and resin was washed with DMF (6×200 mL). This coupling of Fmoc-Nle-OH to modified resin was repeated.

Fmoc was removed by treatment with 200 mL 20% piperidine/DMF for 15 mins, two times. The resin was washed with DMF (6×200 mL).

Assembly of SP-B Fragment 212223 Peptide Chain

Coupling Step—Prepare each of the following Fmoc-amino acids in a solution of 200 mL of DMF: Fmoc-amino acid: Fmoc-Gly-Gly-OH (2.12 g, 6 mmol), Fmoc-Ala-OH (1.86 g, 6 mmol), Fmoc-Val-OH (2.02 g, 6 mmol), Fmoc-Leu-OH (2.12 g, 6 mmol), Fmoc-Pro-OH (2.02 g, 6 mmol), Fmoc-Val-OH (2.02 g, 6 mmol), Fmoc-Val-OH (2.02 g, 6 mmol), Fmoc-Arg(Pbf)-OH (3.88 g, 6 mmol), Fmoc-Cys(Acm)-OH (2.48 g, 6 mmol), Fmoc-Val-OH (2.02 g, 6 mmol), Fmoc-Gln(Trt)-OH (3.66 g, 6 mmol), Fmoc-Ala-OH (1.86 g, 6 mmol), Fmoc-Val-OH (2.02 g, 6 mmol), Fmoc-Ala-OH (1.86 g, 6 mmol), Fmoc-Val-OH (2.02 g, 6 mmol), Fmoc-Ala-OH (1.86 g, 6 mmol), Fmoc-Leu-OH (2.12 g, 6 mmol), Fmoc-Ala-OH (1.86 g, 6 mmol), Fmoc-Gly-OH (1.78 g, 6 mmol), Fmoc-Lys(Boc)-OH (2.82 g, 6 mmol), Fmoc-Pro-OH (2.02 g, 6 mmol), Fmoc-Ile-OH (2.12 g, 6 mmol), Fmoc-Nle-OH (2.12 g, 6 mmol), Fmoc-Ala-OH (1.86 g, 6 mmol), Fmoc-Gln(Trt)-OH (3.66 g, 6 mmol), Fmoc-Ile-OH (2.12 g, 6 mmol), Fmoc-Arg(Pbf)-OH (3.88 g, 6 mmol), Fmoc-Lys(Boc)-OH (2.82 g, 6 mmol), Fmoc-Ile-OH (2.12 g, 6 mmol), Fmoc-Leu-OH (2.12 g, 6 mmol), Fmoc-Ala-OH (1.86 g, 6 mmol), Fmoc-Arg(Pbf)-OH (3.88 g, 6 mmol), Fmoc-Cys(Acm)-OH (2.48 g, 6 mmol), Fmoc-Leu-OH (2.12 g, 6 mmol), Fmoc-Trp(Boc)-OH (3.14 g, 6 mmol), Fmoc-Cys(Acm)-OH (2.48 g, 6 mmol), Fmoc-Tyr(tBu)-OH (2.74 g, 6 mmol), Fmoc-Pro-OH (2.02 g, 6 mmol), Fmoc-Leu-OH (2.12 g, 6 mmol), Fmoc-Pro-OH (2.02 g, 6 mmol), Fmoc-Ile-OH (2.12 g, 6 mmol), Fmoc-Pro-OH (2.02 g, 6 mmol), Fmoc-Phe-OH (1.6 g, 6 mmol).

Reagent a: HBTU (2.27 g, 6.0 mmol); Reagent b: DIPEA (2 mL, 12.0 mmol)

Each Fmoc-amino acid was combined sequentially with reagents a, and b and the resin. Each mixture was mixed and shaken for a minimum of 1.5 hr. The reaction vessel was drained and resin was washed with DMF (6×200 mL).

Coupling efficiency was determined using the ninhydrin test:
i. If the ninhydrin solution was negative (colorless), the next Fmoc amino acid was added as described supra.
ii. If the ninhydrin solution turned positive (blue), the same Fmoc-amino acid was re-coupled as described above. Coupling time was increased, if necessary.

p-nitrophenyl chloroformate (2.4 g, 12 mmol) dissolved by DCM (200 mL) was added into the resin, shaken about 1 hour. Reaction vessel was drained and resin was washed with DMF (3×200 mL). This step was repeated for each successive amino acid to be added.

Recovery of the 212223 Peptide Fragment-Resin

A solution of DIPEA (20 mL) in DMF (150 mL) was added to the resin, and the reaction mixture shaken for 30 min. The reaction vessel was drained and the resin washed with DMF (3×200 mL). The resin was washed with MeOH (2×200 mL) and diethyl ether (2×200 mL), then dried to retrieve the 212223 peptide fragment-resin (20.0 g, 2.0 mmol).

Cleavage of the 212223 Peptide from Resin and Removal of Protecting Groups

The 212223 peptide-resin (20.0 g, 2 mmol) was mixed with cold cleavage solution (TFA/Tris/H2O=95%/2.5%/2.5%, v/v/v; 250 mL) and reaction proceeded in the cold (2 to 8° C.) for 2 hours with gentle shaking. The 21223 peptide-resin was filtered and washed twice with TFA. The filtrates were combined, and 10-fold volume of cold diethyl ether was added. The precipitated peptide was centrifuged and washed with cold diethyl ether three times. The peptide was dried under reduced pressure to obtain crude 212223 peptide (7.5 g).

Preparative HPLC Purification of SP-B Fragment 212223

The crude peptide was purified by prep-HPLC (Mobile Phase: A: 0.05% TFA in water, B: 0.05% TFA in ACN). 540 mg of white solid as a TFA salt was obtained (overall yield: 7.2%) as follows:
Column: Welch Topsil C18, 21.2×250 mm, 5 μm, 150 Å
Loading: 7.5 g of crude peptide in 400 mL of 50% ACN/water
Mobile Phase: A: 0.05% TFA in Water; B: 0.05% TFA in ACN
Gradient: 5% B for 3 min, 40-50% B within 20 min, 50-95% B within 0.5 min, 95% B for 5.5 min. Flow Rate: 25 mL/min.

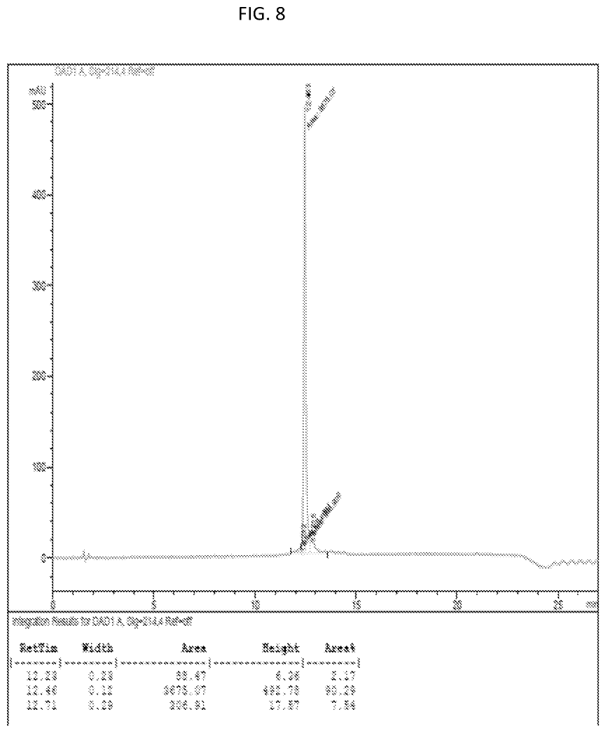

The HPLC-UV-MS analysis of purified fragment 212223 is illustrated in FIG. 8. The X-axis indicates time after sample injection in minutes. Effluent was monitored by UV Spectrophotometry (214 nm). A single quadrupole mass spectrometer was used to verify the mass of Fragment 212223.

LC-MS (ESI) m/z: 1298.0 [M+4H]/4⁺, 1037.9 [M+5H]/5⁺, 865.4 [M+6H]/6⁺.
HPLC: RT: 13.91, purity 91.23% (214 nm).

### Example 3: Peptide Synthesis of Peptide 2122232425-3 by Using Native Chemical Ligation (NCL) Strategy

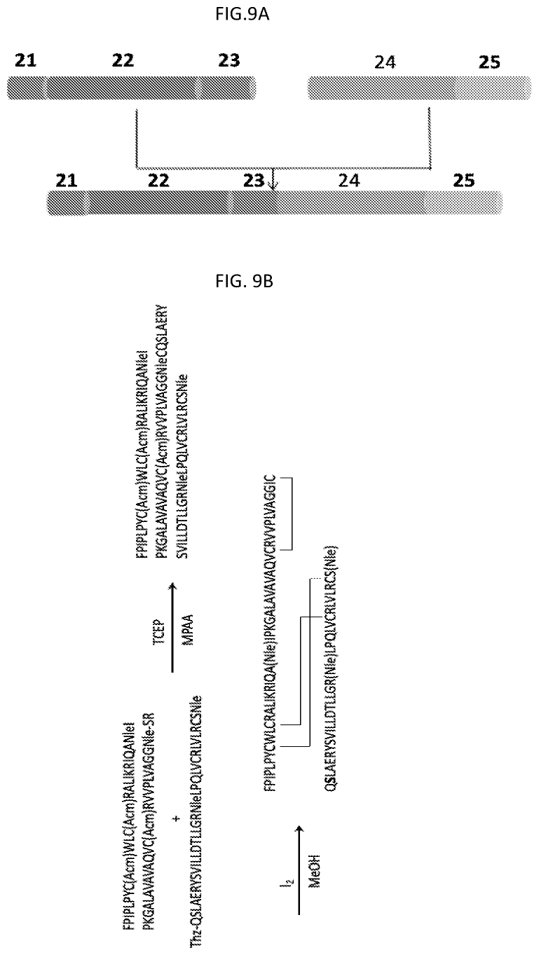

The synthesis scheme is depicted in FIGS. 9A and 9B. The strategy used to assemble full-length monomeric SP-B analog entails: a) The two peptides, 212223 and 2425, were combined using Native Chemical Ligation to form the full-length SP-B monomer. Peptide fragment 212223 (SEQ ID NO:11) consists of 3 regions: 21—residues 1-10, 22—residues 11-34, and 23—residues 35-45. Peptide fragment 2425 consists of 2 regions: 24—residues 46-70, and 25—residues 71-79 as illustrated in FIG. 3. This peptide is labelled 2122232425-3 indicating that the sidechain sulfhydryl of 3 cysteine residues are protected by acetamidomethyl (Acm).

The synthesis used a solution of 6M Gn·HCl buffer (40 mL), MPAA (168 mg, 1 mmol) and TCEP (314 mg, 1.1 mmol), Fragment-212223 (102.0 mg, 0.02 mmol), Fragment-2425 (100 mg, 0.026 mmol) were added. All components were dissolved, solution pH was adjusted to pH 6.8 by the addition of 1 M NaOH in H₂O. The mixture was shaken overnight at room temperature and reaction was monitored by LCMS.

Preparative HPLC Purification of Cvs-Protected SP-B Peptide 2122232425-3

The reaction mixture was purified by prep-HPLC (Mobile Phase: A: 0.05% TFA in water, B: 0.05% TFA in ACN) to get 34 mg of white solid (overall yield: 18.8%) as a TFA salt as follows:
Column: Xbridge Prep C18 (SKU: 186003895; Waters Corporation, Milford, MA 01757), 19×250 mm, 10 μm, 130 Å
Loading: 200 mg of crude peptide in 40 mL of Gn·HCl
Mobile Phase: A: 0.05% TFA in water; B: 0.05% TFA in ACN
Gradient: 38% B for 0.5 min, 38-53% B within 2.5 min, 53-63% B within 20 min, 63-95% B within 0.5 min, 95% B for 5.5 min. Flow Rate: 25 mL/min

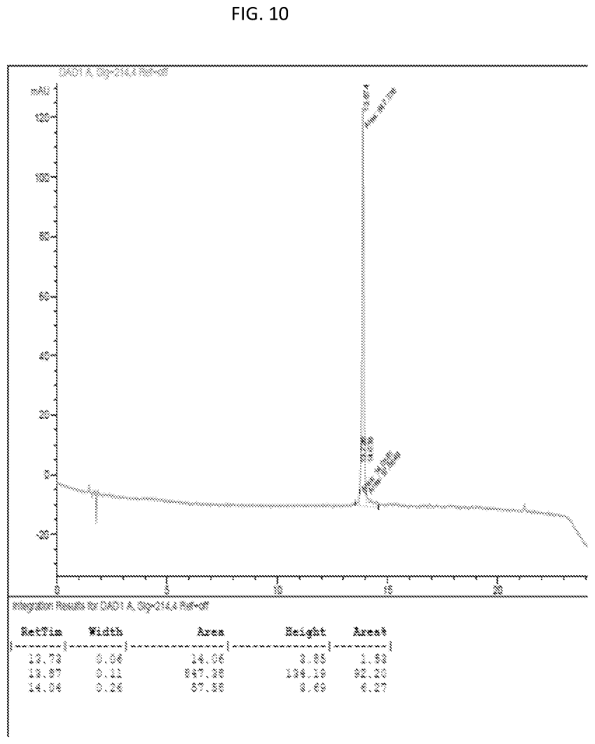

The HPLC profile of Cys-Protected SP-B Fragment 2122232425-3 is illustrated in FIG. 10. The X-axis indicates time after sample injection in minutes. Effluent was monitored by UV Spectrophotometry (214 nm). An electrospray single quadrupole mass spectrometer was used to verify the mass of Fragment 2122232425-3.

LC-MS (ESI) m/z: 744.8 [M+12H]/12⁺, 812.2 [M+11H]/11⁺, 893.7 [M+10H]/10⁺, 992.5 [M+9H]/9⁺, 1116.2[M+8H]/8⁺, 1275.7[M+7H]/7⁺.

HPLC: RT: 17.71, purity 91.15% (214 nm).

### Example 4: Removal of Cystein Sidechain Protection and Oxidation to Monomeric SP-B Peptide

The synthesis scheme added 2122232425-3 (20.0 mg, 1 mmol), to a H₂O/ACN mixture buffer (20 mL) in a flask, pH was adjusted to pH4.0-5.0 with acetic acid. 0.2 mL of 12 in MeOH (0.05 M) was added to the solution and stirred for 35 min. The reaction was monitored by LCMS. The reaction was quenched by adding ascorbic acid.

Preparative Purification of Monomeric [Nle-21, Ser-48,Nle-71,Nle-79]-SP-B

The reaction mixture was purified by prep-HPLC (Mobile Phase: A: 0.05% TFA in water, B: 0.05% TFA in ACN). 2.0 mg of white solid as a TFA salt with 87.88% purity and 1.3 mg with 78% purity were obtained (overall yield: 10.0%) as follows:
Column: Xbridge Prep C18, 19×250 mm, 10 μm, 130 Å
Loading: 20.0 mg of crude peptide in 20 mL of 10% ACN in water
Mobile Phases: A: 0.05% TFA in water; B: 0.05% TFA in ACN
Gradient: 5% B for 3 min, 5-95% B within 20 min, 95% B for 6 min.

Flow Rate: 25 mL/min
Monitored by UV Absorbance (214 nm) and a single quadrupole mass spectrometer using electrospray.

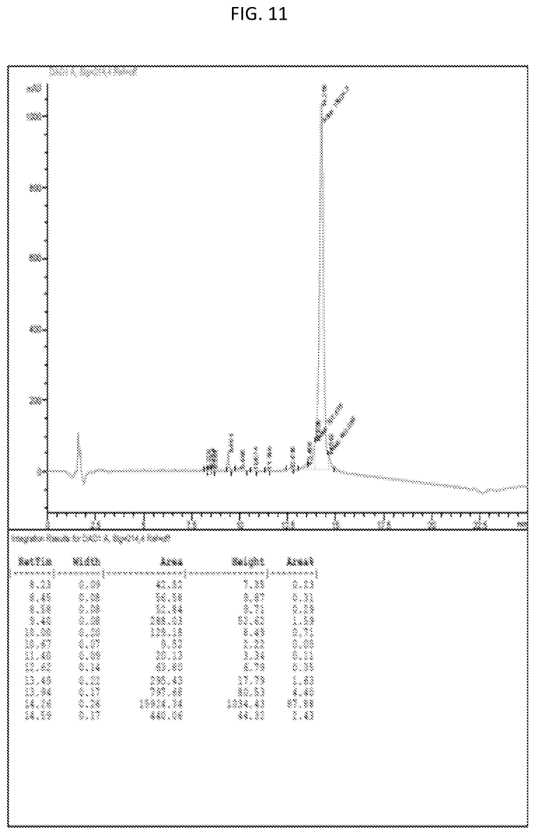

The HPLC Profile of monomeric SP-B Fragment 2122232425 is illustrated in FIG. 11. The X-axis indicates time after sample injection in minutes. Effluent was monitored by UV Spectrophotometry (214 nm). An electrospray single quadrupole mass spectrometer was used to verify the mass of Fragment 2122232425-3.

LC-MS (ESI) m/z: 960.5 [M+9H]/9+, 1080.3 [M+8H]/8+, 1234.7[M+7H]/7+, 1439.8 [M+6H]/6+.

HPLC: RT: 14.26, purity 87.88% (214 nm).

### Example 5: Surface Activity of SP-B Phospholipid Mixture

The synthetic Monomeric SP-B peptide was dissolved in chloroform:methanol:formic acid solutions (9:9:0.5) containing DPPC:POPC:PG (40:30:30). After mixing, solvent was removed by evaporation and the film reconstituted in 10 mM MOPS buffer containing 120 mM NaCl (pH 7.0), 1.5 mM CaCl₂). This mixture was heated to for 10 min. and vortexed for 30 sec. Droplets (10 μL per analysis) contained concentration of SP-B was 0.8 mg/mL and 2 mg/mL phospholipid. Dynamic surface tension was measured using a constrained sessile drop surfactometer (CDS). These droplets were subjected to 20 cycles of compression and expansion. Compression was to ˜40% of the initial droplet size. Each sample was analyzed 4-5 times.

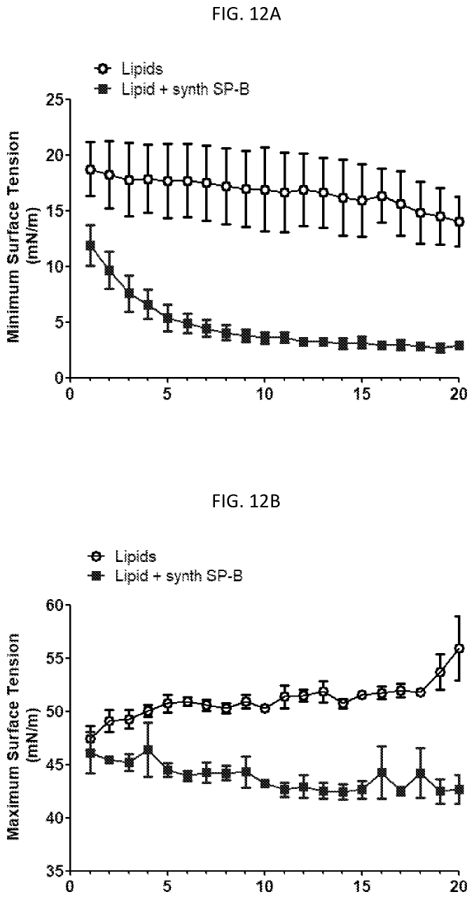

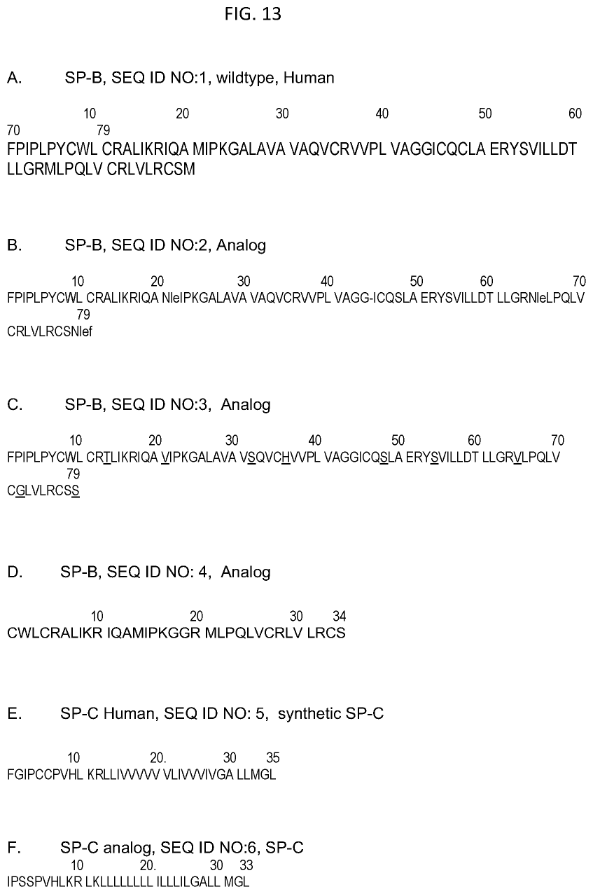

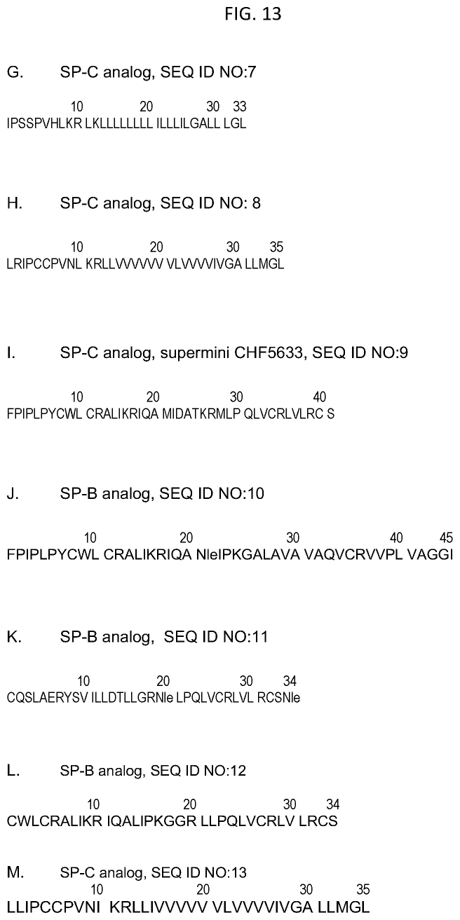

FIGS. 12A and 12B illustrate the dynamic surface tension of Monomeric SP-B peptide in the presence of phospholipid determined with a constrained sessile drop surfactometer. Droplets (10 uL) were alternately constrained and expanded. The X-axis tracks with each successive compression-expansion cycle.

### Example 6 Testing of TAT-FAKp Complexed to or Attached to SP-C

Objective: Evaluate the barrier protective protein-protein interactions induced by TAT-FAKp in endothelial monolayers and mouse lungs. Any one of the TAT peptides found in FIG. 2 can be tested in the procedure provided in U.S. Provisional Patent Application No. 63/092,625.

Mutant TAT-FAKp+SP-C+Mutant TAT-FAKp proteins and mutant FAKp attached to SP-C with non-Mutant TAT-FAKp as a control can be cultured in human lung microvascular, pulmonary artery endothelial cells, and in mouse lungs. In the presence of these mutations, protein-protein interactions and cadherin stability will be determined as reflected by studies of fluorescence recovery after photo bleaching and by assays of endothelial permeability as reflected by the filtration coefficient (Kf), lung water in mice, point permeability assays and the transendothelial resistance (TER), transmigration of leukocytes, and in cultured ECs. Protective effects can be determined using blinded mouse survival studies.

Predicted Results: It is anticipated that loading endothelia with control TAT-FAKp, mutant TAT-FAKp, Mutant TAT-FAKp+SP-C+Mutant TAT-FAKp will abrogate thrombin or oxidant-induced hyperpermeability in lung endothelial monolayers, and ALI-induced lung hyperpermeability in mice. Mouse survival following ALI will be considerably more extended in mutant TAT-FAKp, TAT-FAKp, Mutant TAT-FAKp+SP-C+Mutant TAT-FAKp-treated than in untreated mice. For the first time, the positive therapeutic effects TAT-FAKp will be understood in terms of critical protein-protein interactions induced by activated FAK in conjunction with SP-C.

### Example 7 Testing of Anti-Infective Peptide LL-37 Fused to SP-C

Objective: Evaluate the antimicrobial protective protein-protein interactions induced by SP+C-LL-37 in bronchial mucosal epithelia lung cells.

SP-C+LL37 fusion protein with SP-C peptide as a control can be cultured in infected human lung bronchial mucosal endothelial cells and in mouse lungs. In the presence of SP-C+LL37, protein-protein interactions and cathelicidin activity will be determined as reflected by studies of fluorescence recovery after photo bleaching and by assays of endothelial permeability as reflected by the filtration coefficient (Kf), lung water in mice, point permeability assays and the transendothelial resistance (TER), transmigration of leukocytes, and in cultured ECs. Protective effects can be determined using blinded mouse survival studies.

Predicted Results: It is anticipated that treating infected lung bronchial mucosal endothelia with control SP-C, and SP-C+LL37 fusion protein by for e.g., inter trachiol-intubation will activate the innate immune system in mice as seen by the presence of monocytes, dendritic cells, T cells at the site of microbial infection. Additionally, pathological examination of the infected tissue will exhibit disintegration (damaging and puncturing) of cell membranes where the SP-C+LL37 peptide is active. Mouse survival following lung infection will be considerably more extended in SP-C+LL37 peptide treated than in untreated mice. Additional assays can be found in Kosciuczuk, E. M. et al., “Cathelicidins: family of antimicrobial peptides. A review” (2012) Mol. Biol. Rep. 39(12):10957-10970. [doi: 10.1007/s11033-012-1997-x].

### Example 8 Testing of Anti-Infective Peptide Interferon-v (WWI) Fused to SP-B in the LSC

Objective: Evaluate the antiviral protective protein-protein interactions induced by SP+B+IFNγ in bronchial mucosal epithelia lung cells.

SP-B+IFNγ protein LSC with 85.0:5.0:10.0 DPPG:POGO:SP-B, IFNγ, or SP-B-IFNγ. Using lambs to be delivered at 124 days gestation age, known to have lung immaturity and surfactant deficiency. Lambs will be divided into three groups for LSC treatment: SP-B LSC, IFNγ+SP-B LSC and IFNγ LSC and treated at 15 min. of age. Dosage for SP-B and IFNγ+SP-B will be 200 mg/kg (2.5 ml/kg body weight) and for IFNγ 100 mg/kg (2.0 ml/kg) with 5 lambs in each group. Similar body weights (2.8±0.1 kg) and cord blood pH (7.40±0.03) were between groups.

Predicted Results: Monitoring heart rate, blood pressure, hematocrit, sodium calcium and potassium will be recorded from blood samples collect every 30 min. It is believed that these data points will be less than normal in the SP-8 and IFNγ groups but close to normal values would be anticipated in the IFNγ fused to the N′terminus of SP-B group. The inclusion of ventilation is known to be inductive of infection and/or inflammatory pathological processes and the presence of IFNγ would be expected to mitigate such pathologies in each of the SP-8 and SP-B-IFNγ groups as absence of SP-8, which is needed for maintenance and functioning of the surfactant surface film to reduce surface tension at the alveolar surface for gas exchange Examination for total number of inflammatory cells, monocytes and neutrophils would be expected to be low due to the gentle ventilation protocol followed which minimizes lung stretch.

Additionally, gross and pathological examination of the lungs postmortem will exhibit normal lung expansion where the SP-B LSC, IFNγ+SP-B LSCs would be active. Lamb survival following lung infection will be considerably more extended in both SP-B LSC and IFNγ+SP-B LSC treated groups than in the IFNγ LSC group due to lack of surfactant spreading because of the absence of SP-B. Further data and pathology observations that can be made are found in Sato and Ikegami, (2012) “SP-B and SP-C Containing New Synthetic Surfactant for Treatment of Extremely Immature Lamb Lung” PLOS|One doi.org/10.1371/journal.pone.0039392.

While the principles of this invention have been described in connection with specific embodiments, it should be understood clearly that these descriptions are made only by way of example and are not intended to limit the scope of the invention. What has been disclosed herein has been provided for the purposes of illustration and description. It is not intended to be exhaustive or to limit what is disclosed to the precise forms described. Many modifications and variations will be apparent to the practitioner skilled in the art. What is disclosed was chosen and described in order to best explain the principles and practical application of the disclosed embodiments of the art described, thereby enabling others skilled in the art to understand the various embodiments and various modifications that are suited to the particular use contemplated. It is intended that the scope of what is disclosed be defined by the following claims and their equivalence.

1.-53. (canceled)

54. A polypeptide comprising an amino acid sequence with at least about 95% sequence identity to SEQ ID NO: 1 and, relative to SEQ ID NO: 1, amino acid substitutions M21Nle and M65Nle.

55. The polypeptide of claim 54, comprising, relative to SEQ ID NO: 1, an amino acid substitution of M79Nle.

56. The polypeptide of claim 54, comprising, relative to SEQ ID NO: 1, an amino acid substitution of C48S.

57. A polypeptide comprising an amino acid sequence that has at least about 95% sequence identity to SEQ ID NO: 2 and Nle at amino acid positions 21 and 65.

58. The polypeptide of claim 57, further comprising Nle at amino acid position 79.

59. The polypeptide of claim 57 consisting of SEQ ID NO: 2.

60. A polypeptide comprising an amino acid sequence with at least about 95% sequence identity to SEQ ID NO: 1 and, relative to SEQ ID NO: 1, one or more amino acid substitutions selected from the substitutions in groups (i)-(vii):
(i) A13T, A13S, A13E;
(ii) M21V, M21L, M21I, M21A, M21P, M21Nle;
(iii) A32G, A32S, A32E, A32D;
(iv) C48T, C48E, C48D; (v) S54T, S54A;
(vi) M65V, M65L, M65I, M65A, M65P; and
(vii) M79L, M79S, M79N, M79T, M791, M79A, M79Q, M79P.

61. The polypeptide of claim 60, wherein the amino acid sequence with at least about 95% sequence identity to SEQ ID NO: 1 is SEQ ID NO: 3.

62. A lung surfactant composition, comprising:
(i) a polypeptide comprising an amino acid sequence with at least about 95% sequence identity to SEQ ID NO: 1 and, relative to SEQ ID NO: 1, amino acid substitutions M21Nle and M65Nle; or
(ii) a polypeptide comprising an amino acid sequence that has at least about 95% sequence identity to SEQ ID NO: 2 and Nle at amino acid positions 21 and 65; and
a lipid.

63. The composition of claim 62, further comprising a surfactant protein C (SP-C) polypeptide.

64. The composition of claim 63, wherein the SP-C polypeptide consists of an amino acid sequence with at least about 95% sequence identity to an SP-C polypeptide selected from the group consisting of SEQ ID NO:5, SEQ ID NO: 6, SEQ ID NO: 7, SEQ ID NO: 8, SEQ ID NO: 13.

65. The composition of claim 62, wherein the lipid is a phospholipid.

66. The composition of claim 65, wherein the phospholipid is selected from the group consisting of natural and synthetic phosphoglycerolipids, sphingolipids and digalactosylglycerolipids.

67. The composition of claim 62, wherein the lipid is selected from the group consisting of dipalmitoylphosphatidylcholine (DPPC), 1,2-Dipalmitoyl-sn-glycero-3-phosphoethanolamine (DPPE), 1,2-dipalmitoyl-sn-glycero-3-phosphoglycerol (DPPG), dioleoyl phosphatidylcholine, (DOPC), 1,2-Dioleoyl-sn-glycero-3-phosphocholine, palmitoyloleoylphosphatidylcholine (POPC), palmitoyloleoylphosphatidylglycerol (POPG), phosphaltidylcholine (PC), lysophosphatidylcholine (LPC), phosphatidylethanolamine (PE), lysophosphatidylethanolamine (LPE), phosphatidylglycerol (PG), lysophosphatidylglycerol (LPG), phosphatidylinositol (PI), lysophosphatidylinositol (LPI), phosphatidylserine (PS), lysophosphatidylserine (LPS), cholesterol, palmitoyl-2-oleoyl-sn-glycero-3-phosphoethanolamine (POPE), 1-palmitoyl-2-oleoyl sn-glycero phosphocholine (POPS), 1,2-Distearoyl-sn-glycero-3-phosphocholine (DSPC), palmitoleic, oleic, ricinoleic, vaccenic, linoleic, alpha-linolenic, gamma-linolenic, gadoleic, arachidonic, erucic, sphingomyelin (SM), sphingosine, ceramide, cerebroside, ganglioside, SM with an amine, SM with a quaternary amine, SM that incorporates phosphocholine, SM that includes a phosphoethanolamine, dilauroylphosphatidylcholine (DLPC), disteroylphosphatidylcholine (DSPC), behenoylphosphatidyl-choline, arachidoylphosphatidylcholine (ADPC), [(+)-trimethyl(3-phosphonopropyl)ammonium, mono(2-hexadec-9-enyloxy-3-hexadecyloxypropyl) ester] (DEPN-8), and diether phosphono-phosphatidylglycerol (PG-1).

68. The composition of claim 62, further comprising a buffer that is comprised of a salt and its conjugate acid, wherein the conjugate acid is selected from the group consisting of acetic acid, phosphoric acid, citric acid, boric acid, histidine, lactic acid, tromethamine, gluconic acid, aspartic acid, glutamic acid, tartaric acid, succinic acid, malic acid, fumaric acid, and alpha-ketoglutaric acid.

69. The composition of claim 62, further comprising a bioactive agent.

70. The composition of claim 62, further comprising an excipient selected from the group consisting of a polyol, a sugar, and an amino acid.

71. The composition of claim 70, wherein the polyol is selected from the group consisting of one or more of erythritol, inulin, lactitol, maltitol, mannitol, myoinositol, sorbitol, xylitol and hydrates thereof.

72. The composition of claim 70, wherein the sugar is selected from the group consisting of dextran, fructose, galactose, glucose, hydroxethyl starch, inulin, maltose, raffinose, melezitose, stachyose, sucrose, trehalose, starch, and hydrates thereof.

73. The composition of claim 70, wherein the amino acid is selected from the group consisting of phenylalanine, cystine, glycine, arginine, histidine, isoleucine, lysine, and leucine, proline, methionine, threonine, tryptophan, valine, glutamic acid, aspartic acid, asparagine, glutamine, tyrosine, serine, alanine, tri-leucine, ornithine and salts thereof.

74. The composition of claim 62, wherein the lung surfactant is a dried powder.

75. A method of treatment, comprising administering a composition according to claim 54.
